(19) United States
(12) Patent Application Publication
(10) Pub. No.: US 2025/0034613 A1
(43) Pub. Date: Jan. 30, 2025

(54) ENGINEERED PROBIOTICS FOR DETECTION OF GASTROINTESTINAL DISEASE IN HUMANS

(71) Applicant: Northwestern University, Evanston, IL (US)

(72) Inventors: Arthur Benjamin Prindle, Evanston, IL (US); Jonathan Xia, Evanston, IL (US)

(21) Appl. No.: 18/775,012

(22) Filed: Jul. 17, 2024

Related U.S. Application Data
(60) Provisional application No. 63/528,789, filed on Jul. 25, 2023.

(51) Int. Cl. C12Q 1/02; C12N 1/20; C12R 1/19

(52) U.S. Cl. CPC C12Q 1/02; C12N 1/20; C12R 2001/19; G01N 2800/065

(57) ABSTRACT

Provided herein are engineered bacterial biosensors and methods of use thereof for detection of gastrointestinal disease, including inflammatory bowel disease.

### PRIORITY STATEMENT

This application claims priority to U.S. Provisional Application No. 63/528,789, filed Jul. 25, 2023, the entire contents of which are incorporated herein by reference for all purposes.

### SEQUENCE LISTING

The text of the computer readable sequence listing filed herewith, titled “NWEST_42209_202_SequenceListing.xml”, created Jul. 16, 2024, having a file size of 20,697 bytes, is hereby incorporated by reference in its entirety.

### FIELD

Provided herein are engineered bacterial biosensors and methods of use thereof for methods of detecting gastrointestinal disease. In some aspects, provided herein are engineered bacterial biosensors and methods of use thereof for surveillance of inflammatory bowel disease.

### BACKGROUND

Methods for early and accurate diagnosis of gastrointestinal disease are needed to prevent damage to the underlying tissue. For example, IBD is a spectrum of chronic autoimmune inflammatory disorders of the gastrointestinal tract ranging from ulcerative colitis to Crohn's disease. Globally, prevalence of IBD has been steadily increasing from 3.7 million in 1990 to 6.8 million in 2017, particularly in regions with historically low rates and more limited health care resources. IBD is characterized by a progressive chronic relapsing and remitting course of diarrhea, rectal bleeding, and abdominal pain that can lead to irreversible bowel damage and colorectal cancer if relapses are not identified early. Since the clinical course of IBD varies from patient to patient, the optimal method for monitoring disease activity is difficult. Patient reported symptoms are poor predictors of disease activity. Moreover, disease flares can be difficult to predict and the current practice of IBD disease activity surveillance through endoscopy is invasive and requires medical expertise. Accordingly, what is needed are accurate, non-invasive methods for detection of gastrointestinal disease, such as for IBD surveillance.

### SUMMARY

In some aspects, provided herein are engineered bacterial biosensors of gastrointestinal disease. In some embodiments, the engineered bacterial biosensors comprise a bacterial host cell expressing a reporter molecule operably linked to a calprotectin-responsive promoter. In some embodiments, the engineered bacterial biosensors express a polynucleotide encoding the reporter molecule operably linked to the calprotectin-responsive promoter. In some embodiments, the reporter molecule produces a detectable signal when levels of calprotectin in an environment containing the engineered bacterial biosensor are equal to or above a threshold value. In some embodiments, the bacterial host cell comprises a probiotic bacterial strain. For example, in some embodiments the bacterial host cell comprises a probiotic E. coli strain, such as E. coli Nissle 1917 (EcN).

In some embodiments, the reporter molecule comprises a fluorescent reporter molecule, a bioluminescent reporter molecule, a colorimetric reporter molecule, a CT-detectable reporter molecule, an MRI-detectable reporter molecule, or an ultrasound-detectable reporter molecule.

In some embodiments, the calprotectin-responsive promoter comprises ykgMO. In some embodiments, the calprotectin-responsive promoter is encoded by a polynucleotide having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity) to SEQ ID NO: 1, SEQ ID NO: 2, SEQ ID NO: 4, or SEQ ID NO: 5.

The gastrointestinal disease can be any gastrointestinal disease/disorder or infection affiliated with increased calprotectin production. In some embodiments, the gastrointestinal disease comprises inflammatory bowel disease. In some embodiments, the gastrointestinal disease comprises a gastrointestinal infection, such as Clostridium difficile. In some embodiments, the gastrointestinal disease comprises Celiac disease.

In some aspects, provided herein are methods comprising contacting a sample obtained from a subject having or suspected of having a gastrointestinal disease with an engineered bacterial biosensor provided herein. Such methods are useful for detecting gastrointestinal disease in the subject.

### BRIEF DESCRIPTION OF THE DRAWINGS

FIGS. 1A-1F show that EcN displays distinct gene expression changes in response to calprotectin. (FIG. 1A) RNA-sequencing was performed on EcN that were treated with calprotectin in either minimal (M9) or complex (LB) media. (FIG. 1B) Heat map showing all the significant (padj <0.05) differentially expressed genes induced by calprotectin in EcN in both M9 and LB media (n=3). (FIG. 1C) Principal component analysis performed on RNA-sequencing data (n=3). (FIG. 1D) Venn diagram depicting overlap in statistically significant differentially expressed genes in EcN treated with calprotectin in LB and M9 media. (FIG. 1E-FIG. 1F) Volcano plot of all the significant (padj <0.05) differentially expressed genes induced by calprotectin in EcN in both M9 and LB media.

FIGS. 2A-2H show identification of the EcN promoter ykgMO as a reliable sensor of calprotectin in both LB and M9 media in vitro. (FIG. 2A) Schematic of the screening process used to test the sensitivity of calprotectin responsive promoters identified from the RNA-sequencing data. (FIG. 2B) Four strains of recombinant EcN were generated using the promoters of the top four calprotectin sensitive genes cloned into GFP expression vectors. GFP expression was quantified by TECAN infinite plate reader after each strain (n=3) was treated with 100 μg/g of calprotectin for 13.8 hours in M9 media. (FIG. 2C) Fold-change of GFP expression of calprotectin treated strains over untreated. (FIG. 2D) GFP expression was quantified by TECAN infinite plate reader after recombinant EcN with GFP expression vectors containing ykgMO (n=3) and zinT (n=3) promoters were treated with 100 μg/g of calprotectin for 13.8 hours in LB media. (FIG. 2E) Fold-change of GFP expression of calprotectin treated strains over untreated. (FIG. 2F) GFP expression was quantified by TECAN infinite plate reader after recombinant EcN with GFP expression vector containing ykgMO-IGS promoter (n=3) were treated with 100 μg/g of calprotectin for 13.8 hours in M9 and LB media. (FIG. 2G) Fold-change of GFP expression of calprotectin treated strains over untreated. (FIG. 2H) A representative flow cytometry image of ykgMO-IGS EcN from 1-3 hours of treatment with 100 μg/g of calprotectin with correcting graph of GFP intensities after calprotectin treatment. **p<0.01 and ***p<0.001 for Student's unpaired t-test for indicated comparisons. Data are represented as mean±SEM.

FIGS. 3A-3E show that Calprotectin-sensing EcN can reliably detect gut mucosal inflammation in vivo in DSS induced colitis mouse model. (FIG. 3A) Mice were gavaged daily with 3% DSS for 7 days to induce colitis. CS EcN-Lux or CS EcN-GFP was gavaged daily for three days prior to IVIS imaging or fecal sample collection. (FIG. 3B) Representative live animal luminescence imaging was performed using IVIS Spectrum Instrument on mice (n=4) that were gavaged CS EcN-Lux compared to controls that were not treated with DSS or not gavaged CS EcN-Lux (n=2). (FIG. 3C) Luminescence was quantified for live animal imaging of mice gavaged with CS EcN-Lux with colitis induced by DSS (n=4) and controls (n=2). (FIG. 3D) Representative flow cytometry images quantifying GFP positive cell populations in mice that were treated with DSS and gavaged CS EcN-GFP and controls. (FIG. 3E) GFP expression was quantified by flow cytometry from stool of mice gavaged with CS EcN-GFP with colitis induced by DSS (n=6) and controls (n=6). **p<0.01 and ***p<0.001 for Student's unpaired t-test for indicated comparisons. Data are represented as mean±SEM.

FIGS. 4A-4E show that Calprotectin-sensing EcN can differentiate stool samples from patients with active IBD and those in remission. (FIG. 4A) Stool samples were collected from patients with active IBD (n=6), IBD in remission (n=5), and no IBD (n=6). CS EcN-GFP was co-cultured with stool samples and GFP was quantified using TECAN infinite plate reader after 12 hours. (FIG. 4B) GFP/OD600 of CS EcN-lux i co-cultured with fecal samples of patients with active IBD (n=6), IBD in remission (n=5), and no IBD (n=6). GFP/OD600 were quantified by TECAN Infinite plate reader immediately after co-culturing and 12 hours after co-culturing. (FIG. 4C) Laboratory measured calprotectin values of patients with active IBD (n=6), IBD in remission (n=5), and no IBD (n=6) plotted with their respective GFP/OD600 intensities. (FIG. 4D) Representative flow cytometry images quantifying GFP positive cell populations in CS EcN-GFP that were co-cultured with fecal samples of patients with no IBD, IBD in remission, and active IBD after 3 hours. (FIG. 4E) % GFP positive cells from flow cytometry time course of CS EcN-GFP after co-culturing with patient samples. *p<0.05 for Student's unpaired t-test for indicated comparisons. Data are represented as mean±SEM.

FIG. 5A-5I show construction and validation of CS EcN-Lux for in vivo inflammation detection in DSS treated mice. (FIG. 5A) Luminescence was quantified by a BioTek Synergy Neo2 Multi-mode plate reader after CS EcN-Lux (n=3) was treated with 100 μg/g of calprotectin for 12.25 hours in M9 media. (FIG. 5B) Maximum Luminescence achieved by CS EcN-Lux after 100 μg/g of calprotectin compared to untreated controls during 12.25 hour growth curve in M9 media. (FIG. 5C) OD600 measurements during 12.25 hour growth curve for CS EcN-Lux (n=3) and CS EcN-Lux (n=3) treated with 100 μg/g of calprotectin in M9 media. (FIG. 5D) Calprotectin dose response curve of CS EcN-Lux strain with varying doses of calprotectin grown in M9 media. (FIG. 5E) Maximum Luminescence achieved by CS EcN-Lux during varying doses of calprotectin grown in M9 media. (FIG. 5F) Representative H&E staining of colons of mice treated with DSS and controls. (FIG. 5G-FIG. 5H) Serum and stool calprotectin levels of mice treated with DSS (n=5) and untreated controls (n=5). (FIG. 5I) Fold change of GFP positive cells present in stool of DSS-treated and control mice that were both gavaged with CS EcN-GFP.

FIG. 6 shows exemplary sequences for the ykgMO promoter and a portion of the superfolder GFP sequence. The ykgMO promoter is encoded by the polynucleotide of SEQ ID NO: 1, and a portion of the superfolder GFP encoded by SEQ ID NO: 6 is shown.

FIG. 7 shows an exemplary sequence for the ykgMO-IGS promoter sequence. The ykgMO-IGS is encoded by the polynucleotide of SEQ ID NO: 2.

FIG. 8 shows an exemplary sequence for the ykgMO-IGS promoter sequence comprising a ribosomal binding site. The promoter is encoded by SEQ ID NO: 5.

FIG. 9 shows an exemplary sequence for the ykgMO promoter comprising a ribosomal binding site. The promoter is encoded by SEQ ID NO: 4.

FIG. 10 shows recombinant EcN treated with fecal samples of patients with CDI (n=2) and no gut infection (n=3). GFP expressions were quantified by TECAN infinite plate reader after 3 and 12 hours. **p<0.01 and ***p<0.001 for Student's unpaired t-test for indicated comparisons. Data are represented as mean±SEM.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). In some embodiments, the subject is a human. In some embodiments, the subject has or is suspected of having inflammatory bowel disease.

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely cures the disease and/or adverse symptom attributable to the disease. “Treatment” may refer to reducing the amount or severity of a particular condition, disease state (e.g., inflammatory bowel disease), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

The term “pharmaceutical formulation” as used herein refers to a composition comprising at least one pharmaceutically-active agent, chemical substance or drug. The pharmaceutical formulation may be in solid or liquid form and can comprise at least one additional active agent, carrier, vehicle, excipient or auxiliary agent identifiable by the skilled person. The pharmaceutical formulation may be in the form of a tablet, capsule, granules, powder, liquid or syrup.

The term “effective dose” or “effective amount” refers to an amount of an agent, e.g., a neutralizing antibody, that results in the reduction of symptoms in a patient, treatment of prevention of a disease or condition, or results in a desired biological outcome.

As used herein, the terms “administration” and “administering” refer to the act of providing an agent (e.g. drug, prodrug, therapeutic, or other agent) to a subject. Suitable routes of administration include, without limitation: topical, parenteral (e.g. by injection), oral, and by inhalation. Exemplary parenteral routes include, but are not limited to, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Inflammatory bowel disease (IBD) is a spectrum of autoimmune diseases affecting the gastrointestinal tract characterized by a relapsing and remitting course of gut mucosal inflammation. Suitable rapid, non-invasive markers for IBD surveillance are lacking. This disclosure addresses this need and provides an engineered probiotic capable of detecting the IBD biomarker calprotectin with high sensitivity and specificity in IBD patients. Specifically, a bacterial promoter was used in the probiotic strain E. coli Nissle 1917 (EcN) which exhibits a specific expression increase in the presence of calprotectin. Using murine models of colitis, reporter signal is activated in vivo during transit of the GI tract following oral delivery. Furthermore, the engineered probiotic successfully discriminates human patients with active IBD from those in remission and those without IBD using patient stool samples, where the intensity of reporter signal quantitatively tracks with clinical laboratory measured levels of calprotectin. The biosensors herein are widely applicable to any gastrointestinal disease associated with mucosal damage and/or calprotectin production.

In some aspects, provided herein are engineered bacterial biosensors. In some embodiments, provided herein are engineered bacterial biosensors of gastrointestinal disease. As used herein, the term “gastrointestinal disease” is used in the broadest sense and is inclusive of any disease/disorder or infection of the gastrointestinal system. In some embodiments, the gastrointestinal disease is characterized by mucosal damage and increased calprotectin production. In some embodiments, the gastrointestinal disease is inflammatory bowel disease. In some embodiments, the gastrointestinal disease is Celiac disease. In some embodiments, the gastrointestinal disease is a gastrointestinal infection, such as Clostridium difficile.

In some embodiments, the engineered bacterial biosensor comprises a bacterial host cell expressing a reporter molecule operably linked to a calprotectin-responsive promoter. In some embodiments, the bacterial host cell expresses a vector (e.g. plasmid) encoding the reporter molecule and the calprotectin-responsive promoter. For example, in some embodiments the bacterial host cell expresses a vector comprising a sequence encoding the reporter molecule and a sequence encoding the calprotectin-responsive promoter, wherein expression of the reporter molecule is operably linked to the calprotectin-responsive promoter. In some embodiments, the reporter molecule produces a detectable signal when levels of calprotectin in an environment containing the engineered bacterial biosensor are equal to or above a threshold value. For example, in some embodiments the reporter molecule produces a detectable signal within the bowel of a subject when the levels of calprotectin in the bowel are equal to or above a threshold value. As another example, in some embodiments the reporter molecule produces a detectable signal in a sample, such as a stool sample, obtained from the subject when the levels of calprotectin within the sample (e.g. within the stool sample) are equal to or above a threshold value.

Any suitable bacterial host cell may be used. In some embodiments, the bacterial host cell comprises a probiotic bacterial strain. As used herein, the term “probiotic” refers to a bacteria that confer a health benefit to the host. In some embodiments, the bacterial host cell comprises a probiotic bacterial strain that colonizes in the human gut. In some embodiments, the bacterial host cell comprises a probiotic strain selected from the genera Lactobacillus, Bifidobacterium, Saccharomyces, Streptococcus, Enterococcus, Escherichia, and Bacillus. In some embodiments, the bacterial host cell comprises a probiotic E. coli strain. In some embodiments, the bacterial host cell comprises E. coli Nissle 1917 (EcN).

The reporter molecule may be any suitable reporter molecule that produces a detectable signal in response to calprotectin (e.g. in response to calprotectin activating the calprotectin-sensitive promoter, thereby inducing expression of the reporter molecule in the host cell). In some embodiments, the reporter molecule comprises a fluorescent reporter molecule, a bioluminescent reporter molecule, a colorimetric reporter molecule, a CT-detectable reporter molecule, an MRI-detectable reporter molecule, or an ultrasound-detectable reporter molecule. An exemplary CT-detectable reporter molecule is thymidine kinase. In some embodiments, the reporter molecule is an MRI-detectable reporter molecule, such as mms6 or MS-1 MagA. In some embodiments, the reporter molecule is a fluorescent reporter molecule. Exemplary fluorescent reporter molecules include, but are not limited to, green fluorescent proteins, blue fluorescent proteins, cyan fluorescent proteins, yellow fluorescent protein, orange fluorescent proteins, red fluorescent proteins, and modified forms or derivatives thereof. In some embodiments, the reporter molecule comprises super folder green fluorescent protein (sfGFP) . . . . In some embodiments, the superfolder GFP is encoded by the sequence

(SEQ ID NO: 6)
ATGAGCAAAGGAGAAGAACTTTTCACTGGAGTTGTCCCAATTCTTGTTG
AATTAGATGGTGATGTTAATGGGCACAAATTTTCTGTCCGTGGAGAGGG
TGAAGGTGATGCTACAAACGGAAAACTCACCCTTAAATTTATTTGCACT
ACTGGAAAACTACCTGTTCCGTGGCCAACACTTGTCACTACTCTGACCT
ATGGTGTTCAATGCTTTTCCCGTTATCCGGATCACATGAAACGGCATGA
CTTTTTCAAGAGTGCCATGCCCGAAGGTTATGTACAGGAACGCACTATA
TCTTTCAAAGATGACGGGACCTACAAGACGCGTGCTGAAGTCAAGTTTG
AAGGTGATACCCTTGTTAATCGTATCGAGTTAAAGGGTATTGATTTTAA
AGAAGATGGAAACATTCTTGGACACAAACTCGAGTACAACTTTAACTCA
CACAATGTATACATCACGGCAGACAAACAAAAGAATGGAATCAAAGCTA
ACTTCAAAATTCGCCACAACGTTGAAGATGGTTCCGTTCAACTAGCAGA
CCATTATCAACAAAATACTCCAATTGGCGATGGCCCTGTCCTTTTACCA
GACAACCATTACCTGTCGACACAATCTGTCCTTTCGAAAGATCCCAACG
AAAAGCGTGACCACATGGTCCTTCTTGAGTTTGTAACTGCTGCTGGGAT
TACACATGGCATGGATGAGCTCTACAAATAA.

In some embodiments, the reporter molecule is a bioluminescent reporter molecule, such as a luciferase. In some embodiments, the reporter molecule is the luciferase encoded by the LuxA gene. In some embodiments, the reporter molecule is a colorimetric reporter molecule. Exemplary colorimetric reporter molecules include, for example, LacZ, GusA, CelB, AES, NagZ, PhoA, and Est2. Exemplary ultra-sound detectable reporter molecules include, for example, ultrasound-sensitive gas vesicles (GVs). In some embodiments, the bacterial host cell expresses a vector (e.g. a plasmid) encoding a reporter molecule, including any of the above-described reporter molecules. Accordingly, in such embodiments the plasmid comprises a sequence encoding the reporter molecule (e.g. encoding the fluorescent reporter molecule, colorimetric reporter molecule, bioluminescent reporter molecule, CT-detectable reporter molecule, MRI-detectable reporter molecule, ultra-sound detectable reporter molecule, etc.)

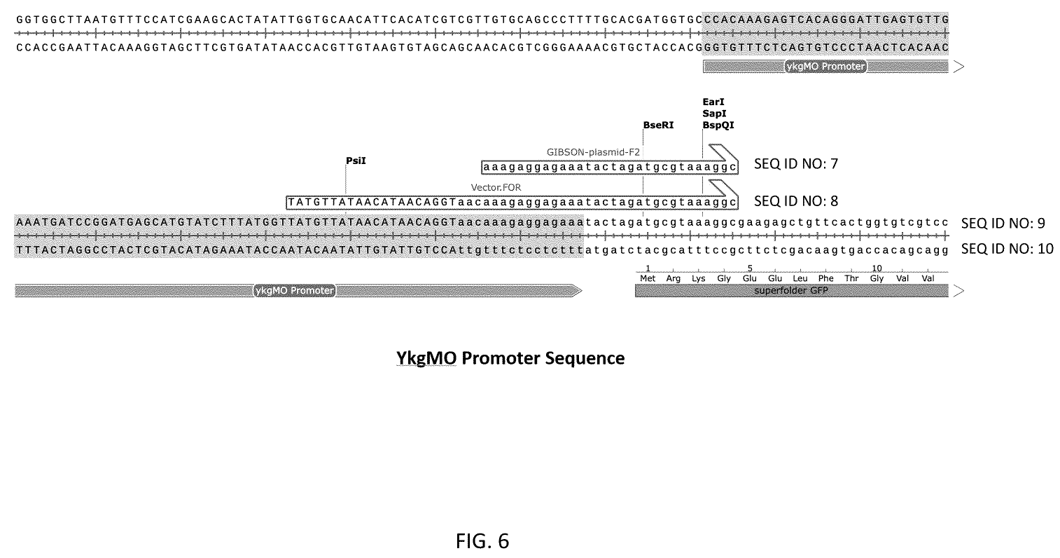

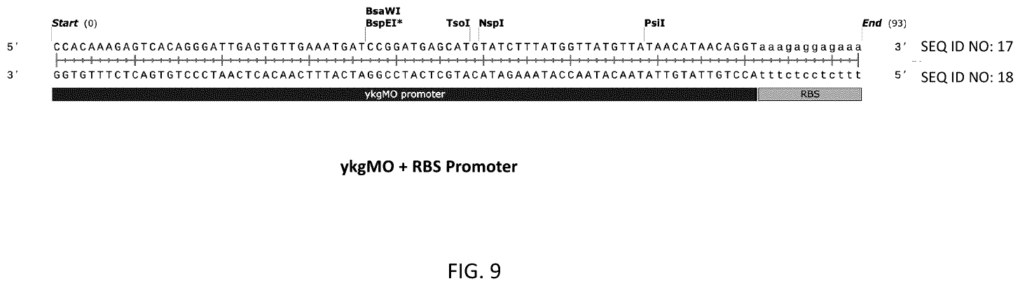

In some embodiments, the reporter molecule is operably linked to a calprotectin-responsive promoter. The calprotectin-responsive (e.g. calprotectin-sensitive) promoter may be a promoter of any calprotectin-responsive gene (e.g. calprotectin-sensitive) gene. A calprotectin-responsive or a calprotectin-sensitive gene are used interchangeably herein and refer to a gene that is differentially expressed in conditions with and without calprotectin or differentially expressed in conditions with and without a threshold level of calprotectin. In some embodiments, a calprotectin-responsive gene is upregulated in response to a sufficient level or amount of calprotectin in contrast to an insufficient level or amount of calprotectin. In some embodiments, the calprotectin-responsive promoter is a promoter from a calprotectin-responsive gene that is naturally (i.e. natively) expressed in the bacterial host cell. For example, in some embodiments the bacterial host cell is E. coli Nissle 1917 (EcN) and the calprotectin-responsive promoter is a promoter from a calprotectin-responsive gene native to E. coli Nissle 1917 (EcN). For example, in some embodiments the bacterial host cell is E. coli Nissle 1917 (EcN) and the calprotectin-responsive promoter is ykgMO. FIG. 6 shows exemplary sequences for the ykgMO promoter, along with exemplary sequences for superfolder GFP, which may be used as the reporter molecule in a biosensor provided herein. In some embodiments, the promoter comprises a sequence set forth in FIG. 6 or FIG. 9.

In some embodiments, the ykgMO promoter is encoded by the sequence: CCACAAAGAGTCACAGGGATTGAGTGTTGAAATGATCCGGATGAGCATGTATCTTTA TGGTTATGTTATAACATAACAGGT (SEQ ID NO: 1). In some embodiments, the ykgMO promoter is encoded by a sequence having at least 80% sequence identity with SEQ ID NO: 1 In some embodiments, the ykgMO promoter is encoded by a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 1. In some embodiments, the promoter is encoded by the sequence of SEQ ID NO: 1 or a sequence having at least 80% identity with SEQ ID NO: 1, and additionally contains a ribosomal binding site (e.g. encoded by SEQ ID NO: 3). In some embodiments, the bacterial host cell expresses a vector comprising a promoter-encoding sequence having at least 80% identity to SEQ ID NO: 1. In some embodiments, the bacterial host cell expresses a vector comprising a promoter-encoding sequence (e.g. a polynucleotide sequence) having at least 80% identity to SEQ ID NO: 1 and additionally contains a sequence encoding a ribosomal binding site, such as SEQ ID NO: 3.

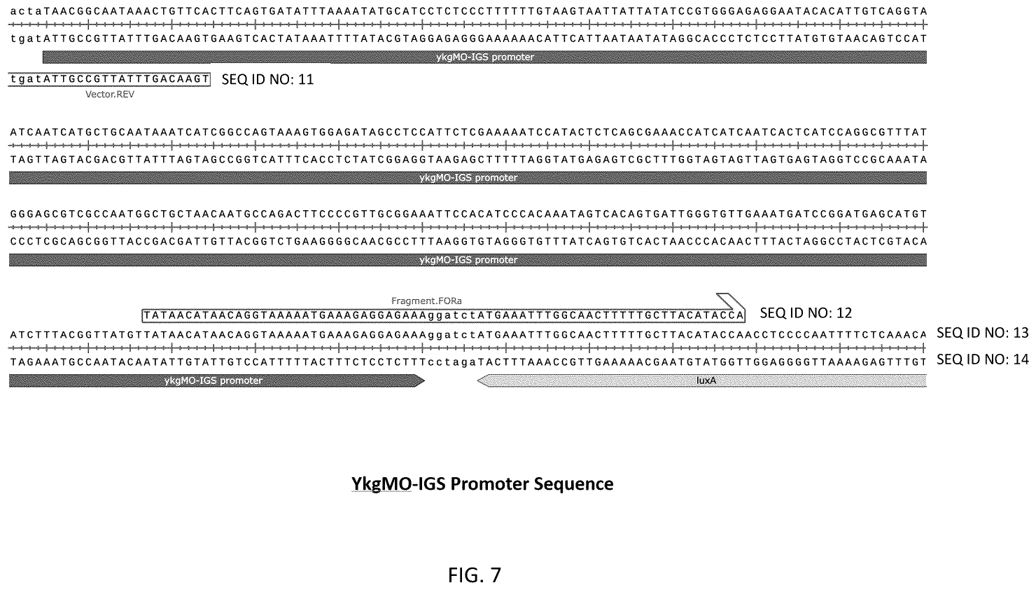

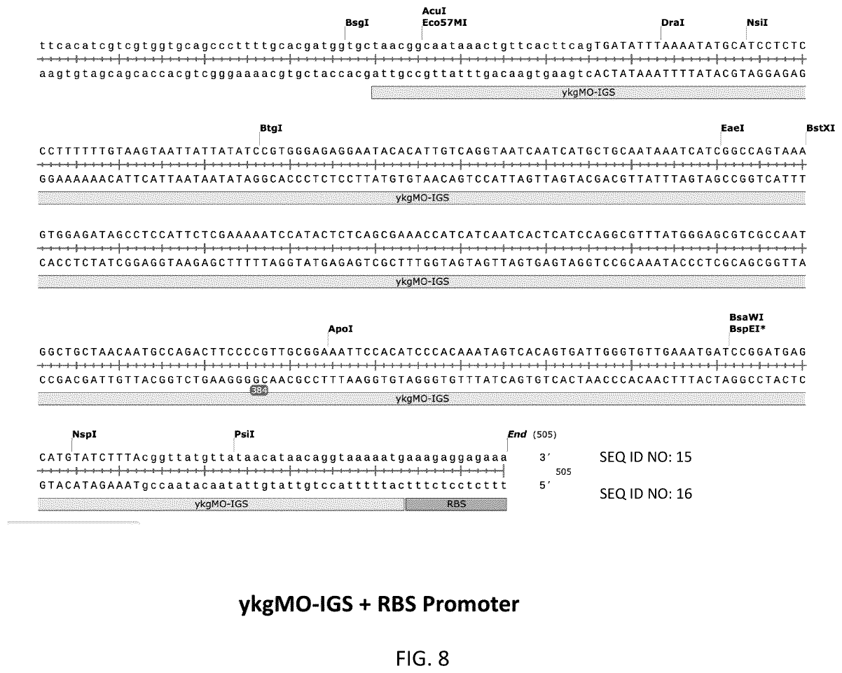

In some embodiments, promoter or the vector (e.g. plasmid) containing the polynucleotide encoding the promoter additionally contains one or more regions of the gene from which the promoter was obtained. For example, in some embodiments the promoter or the polynucleotide encoding the promoter additionally contains intergenic regions upstream of the gene from which the promoter was obtained. An exemplary sequence of the polynucleotide (e.g. promoter-encoding sequence) encoding the ykgMO promoter containing upstream intergenic regions (ykgMO-IGS) is shown in FIG. 7 and FIG. 8. In some embodiments, the promoter comprises ykgMO with intergenic regions (ykgMO-IGS) and is encoded by a polynucleotide having the sequence TAACGGCAATAAACTGTTCACTTCAGTGATATTTAAAATATGCATCCTCTCCCTTTTT TGTAAGTAATTATTATATCCGTGGGAGAGGAATACACATTGTCAGGTAATCAATCAT GCTGCAATAAATCATCGGCCAGTAAAGTGGAGATAGCCTCCATTCTCGAAAAATCC ATACTCTCAGCGAAACCATCATCAATCACTCATCCAGGCGTTTATGGGAGCGTCGCC AATGGCTGCTAACAATGCCAGACTTCCCCGTTGCGGAAATTCCACATCCCACAAATA GTCACAGTGATTGGGTGTTGAAATGATCCGGATGAGCATGTATCTTTACGGTTATGT TATAACATAACAGGTAAAAATG (SEQ ID NO: 2). In some embodiments, the promoter is encoded by a polynucleotide having at least 80% sequence identity with SEQ ID NO: 2. In some embodiments, the promoter is encoded by a polynucleotide comprising a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 2.

In some embodiments, the promoter (e.g. ykgMO promoter) or the polynucleotide encoding the promoter comprises a ribosomal binding site (RBS). Exemplary sequences encoding promoters containing a ribosomal binding site are shown in FIG. 8 and FIG. 9. In some embodiments, the RBS is encoded by the sequence AAAGAGGAGAAA (SEQ ID NO: 3). In some embodiments, the promoter is encoded by a polynucleotide comprising the sequence CCACAAAGAGTCACAGGGATTGAGTGTTGAAATGATCCGGATGAGCATGTATCTTTA TGGTTATGTTATAACATAACAGGTAAAGAGGAGAAA (SEQ ID NO: 4). In some embodiments, the promoter is encoded by a polynucleotide having at least 80% sequence identity with SEQ ID NO: 4. In some embodiments, the promoter is encoded by a polynucleotide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 4.

In some embodiments, the promoter comprises intergenic regions and a ribosomal binding site. For example in some embodiments the promoter is encoded by a polynucleotide comprising the sequence TAACGGCAATAAACTGTTCACTTCAGTGATATTTAAAATATGCATCCTCTCCCTTTTT TGTAAGTAATTATTATATCCGTGGGAGAGGAATACACATTGTCAGGTAATCAATCAT GCTGCAATAAATCATCGGCCAGTAAAGTGGAGATAGCCTCCATTCTCGAAAAATCC ATACTCTCAGCGAAACCATCATCAATCACTCATCCAGGCGTTTATGGGAGCGTCGCC AATGGCTGCTAACAATGCCAGACTTCCCCGTTGCGGAAATTCCACATCCCACAAATA GTCACAGTGATTGGGTGTTGAAATGATCCGGATGAGCATGTATCTTTACGGTTATGT TATAACATAACAGGTAAAAATGAAAGAGGAGAAA (SEQ ID NO: 5). In some embodiments, the promoter is encoded by a polynucleotide having at least 80% sequence identity with SEQ ID NO: 5. In some embodiments, the promoter comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with SEQ ID NO: 5.

The engineered bacterial biosensors provided herein find use in methods of assessing gastrointestinal disease (e.g. inflammatory bowel disease, Clostridium difficile infection, Crohn's disease, etc.) in a subject. In some aspects, provided herein is a method comprising contacting a sample obtained from a subject having or suspected of having a gastrointestinal disease with an engineered bacterial biosensor described herein, and measuring a signal from the reporter molecule in the sample. In some embodiments, provided herein is a method comprising providing an engineered bacterial biosensor described herein to a subject, obtaining a sample from the subject, and measuring a signal from the reporter molecule in the sample. The engineered bacterial biosensor may be provided to the subject by any suitable route, including oral and parenteral routes (e.g. injection, including intravenous, intramuscular, subcutaneous, and the like).

In some embodiments, a signal equal to or above a threshold value indicates that the subject has the gastrointestinal disease, and a signal below a threshold value indicates that the subject does not have the gastrointestinal disease or is in remission from the disease. For example, in some embodiments, a signal equal to or above a threshold value indicates that the subject has active inflammatory bowel disease, and a signal below a threshold value indicates that the subject is in remission or does not have inflammatory bowel disease. In some embodiments, the sample is a stool sample.

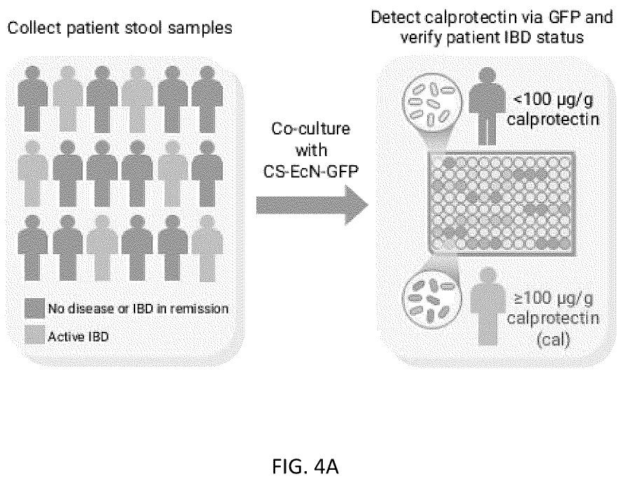

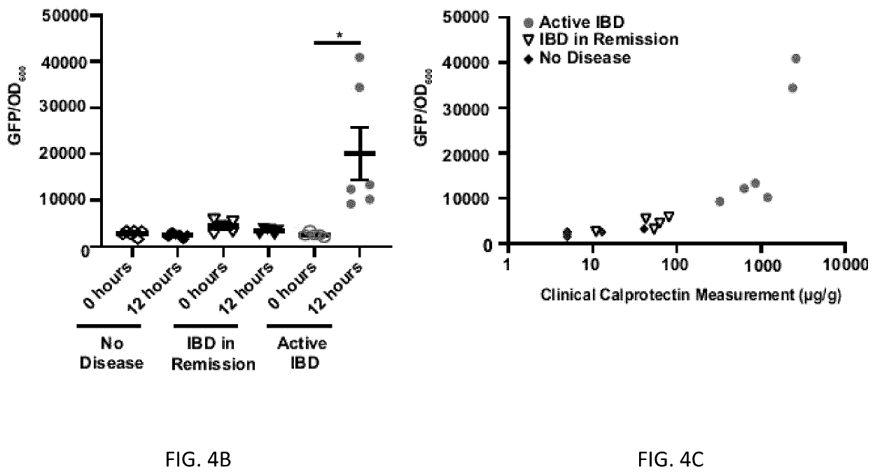

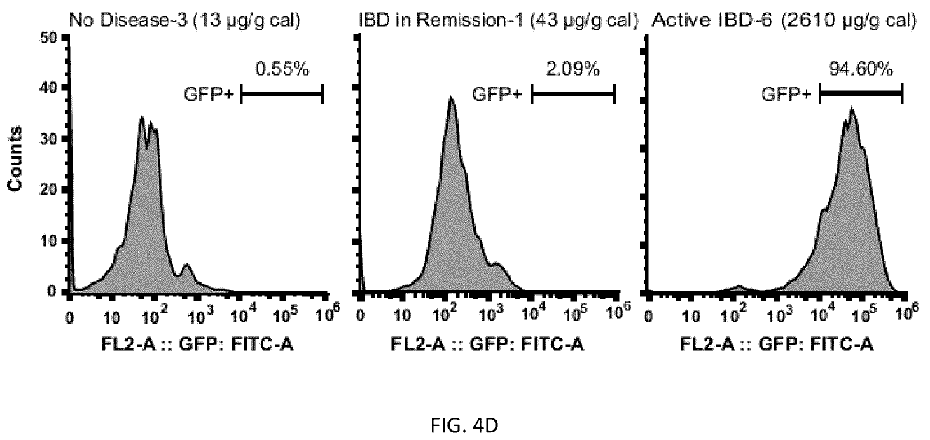

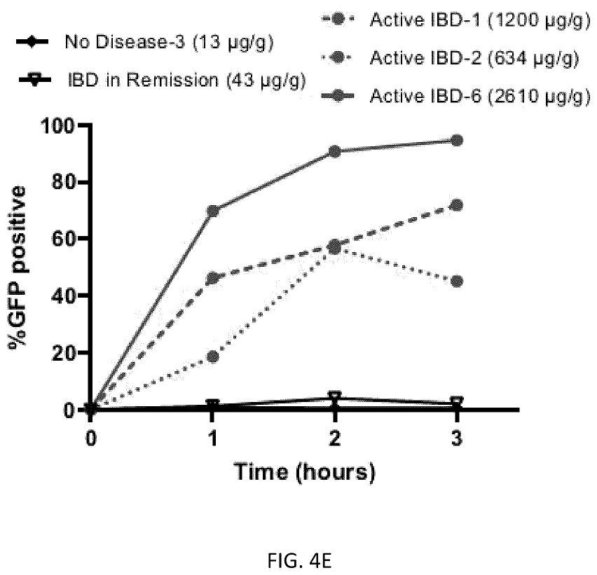

Measuring a signal from the reporter molecule in the sample may be performed by any suitable technique. In some embodiments, the technique depends on the reporter molecule used. For example, a fluorescence, colorimetric, or bioluminescent readout may be measured from the reporter molecule when the reporter molecule is a fluorescent reporter, a colorimetric reporter, or a bioluminescent reporter. The threshold value may be determined, known, or calculated based upon the signal from the reporter molecule in one or more samples obtained from a subject(s) known to not have IBD and/or from a subject(s) known to have IBD. The exact threshold value may depend on the particular technique used to assess the signal from the reporter molecule, and the reporter molecule used. In exemplary methods, the reporter molecule is GFP and a signal from the reporter molecule is measured in a stool sample obtained from the subject using a plate reader. In such embodiments, an exemplary threshold value is 5,000 RFU (see FIG. 4). For example, a signal from the reporter molecule in the sample equal to or above 5,000 RFU indicates that the subject has gastrointestinal disease (e.g. IBD), whereas a signal less than 5,000 RFU indicates that the subject does not have or is in remission from the gastrointestinal disease (e.g. IBD). This is only an exemplary threshold value determined using a specific reporter molecule and a specific plate reader, it is understood that the threshold value will vary depending on the reporter molecule used and the technique used to detect the signal.

In some embodiments, measuring a signal from the reporter molecule is performed using a plate reader. The methods provided herein are advantageous in that affordable equipment with minimal processing steps can be used for efficient, rapid, and sensitive detection of gastrointestinal disease. For example, a plate reader may be used to evaluate a signal (e.g. a fluorescent signal) from the reporter molecule (e.g. GFP fluorescence), and a fluorescent signal equal to or above a threshold value indicates that the subject has the gastrointestinal disease whereas a fluorescent signal below the threshold value indicates that the subject does not have or is in remission from the gastrointestinal disease. The fluorescent signal may be normalized, such as to optical density (e.g. OD).

In some embodiments, measuring a signal from the reporter molecule in the sample comprises assessing the number or percentage of cells in the sample expressing the reporter molecule. For example, the number or percentage of cells expressing the reporter molecule (e.g. the number of GFP positive cells) can be assessed by flow cytometry. A number or percentage of cells equal to or above a threshold value indicates that the subject has the gastrointestinal disease, whereas a number or percentage of cells below a threshold value indicates that the subject is in remission from or does not have the gastrointestinal disease. The threshold value may be determined, known, or calculated based upon the number of cells expressing the reporter molecule in one or more samples obtained from a subject(s) known to not have IBD and/or from a subject(s) known to have IBD. For example, in some embodiments the threshold value for the percentage of cells expressing the reporter molecule is 5-15% (e.g. 5%, 6%, 7%, 8%, 9%, 10%, 11%, 12%, 13%, 14% or 15%). In some embodiments the threshold value for the percentage of cells expressing the reporter molecule is 5-10% (e.g. 5%, 6%, 7%, 8%, 9%, or 10%). In some embodiments, if the number or percentage of cells expressing the reporter molecule is above the threshold value (e.g. about 15%, above 10%) the subject has the gastrointestinal disease, whereas if the number or percentage of cells expressing the reporter molecule is below the threshold value (e.g. below 10%, below 5%, etc.) the subject does not have or is in remission from the gastrointestinal disease.

In some embodiments, provided herein is a method comprising providing an engineered bacterial biosensor described herein to a subject (e.g. a subject having or suspected of having a gastrointestinal disease) and performing an imaging procedure on a subject to detect the signal from the reporter molecule. In some embodiments, the imaging procedure comprises a CT scan, an MRI, or an ultrasound. In some embodiments, the imaging procedure is performed on the subject to visualize a signal within the gut of the subject. In some embodiments, the subject has or is suspected of having a gastrointestinal disease and the imaging procedure is performed to determine whether the subject has the gastrointestinal disease. For example, such an imaging procedure may be performed to determine whether the subject has active IBD, is in remission, or does not have IBD. In some embodiments, a detectable signal equal to or above a threshold value indicates that the subject has active IBD, whereas a signal less than a threshold value indicates that a subject is in remission or does not have IBD. The threshold value may be determined, known, or calculated based upon the signal from the reporter molecule in one or more samples obtained from a subject(s) known to not have IBD and/or from a subject(s) known to have IBD. The exact threshold value may depend on the particular technique used to assess the signal from the reporter molecule, and the reporter molecule used. In some embodiments, the threshold value is the background signal from the reporter molecule present in a control subject not afflicted with the gastrointestinal disease, and a signal greater than the threshold signal (e.g. a signal above background) indicates that the subject has the gastrointestinal disease whereas a signal equal to or below the background signal indicates that the subject does not have or is in remission from the gastrointestinal disease.

Measuring a signal from the reporter molecule in the sample may be performed by any suitable technique. In some embodiments, the technique depends on the reporter molecule used. For example, an imaging procedure is performed when the reporter molecule is an MRI-responsive or a CT-responsive reporter molecule, or an ultrasound is performed when the reporter molecule is an ultrasound-responsive reporter molecule. As another example, a fluorescence, colorimetric, or bioluminescent readout may be measured from the reporter molecule when the reporter molecule is a fluorescent reporter, a colorimetric reporter, or a bioluminescent reporter.

### Experimental

Inflammatory bowel disease (IBD) is a spectrum of chronic inflammatory gastrointestinal diseases that is difficult to monitor due to the relapsing and remitting nature of disease flares often resulting in downstream complications. The standard of disease surveillance through colonoscopy is invasive, costly, and requires medical expertise. C-reactive protein (CRP) is a systemic biomarker that indirectly quantifies disease activity, but it is non-specific to IBD as it is elevated in other infectious and autoimmune illnesses. Calprotectin is a stool biomarker that is both sensitive and specific to gut inflammation, but laboratory results for calprotectin usually take 1-2 weeks to obtain in the clinical setting. Thus, a clinical gap remains in methods of precise, rapid, and non-invasive monitoring of disease activity. To address this gap, the probiotic E. coli Nissle 1917 (EcN) was engineered herein to detect calprotectin to provide a non-invasive diagnostic of gut inflammation. EcN is a human gut colonizing probiotic with proven safety in humans and compatibility with the canonical genetic engineering techniques for bacteria. Calprotectin responsive EcN genes were identified through RNA-sequencing (RNA-seq) and their respective promoters were coupled to a fluorescent reporter prior to transforming into EcN. Next, these engineered EcN sensors were evaluated for sensitivity and specificity to calprotectin and performance was optimized. Finally, it was demonstrated that the engineered EcN sensor is sensitive and specific to gut inflammation in murine models of IBD and stool samples from human patients with active IBD. Accordingly, the engineered probiotics provided herein can be used for IBD monitoring in real-time. Real-time monitoring can be used to identify at-risk patients early with the goal of maintaining remission and avoiding irreversible bowel damage in this growing patient population.

### Results

Calprotectin Treatment Results in Robust Transcriptional Changes in E. coli Nissle 1917

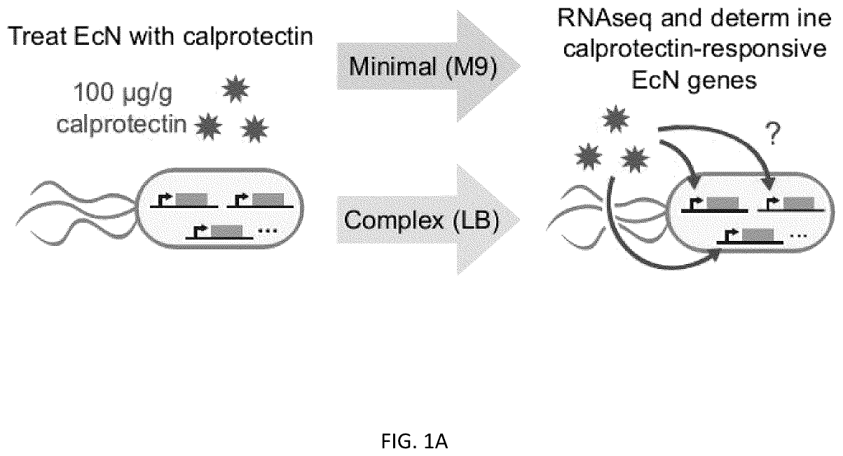

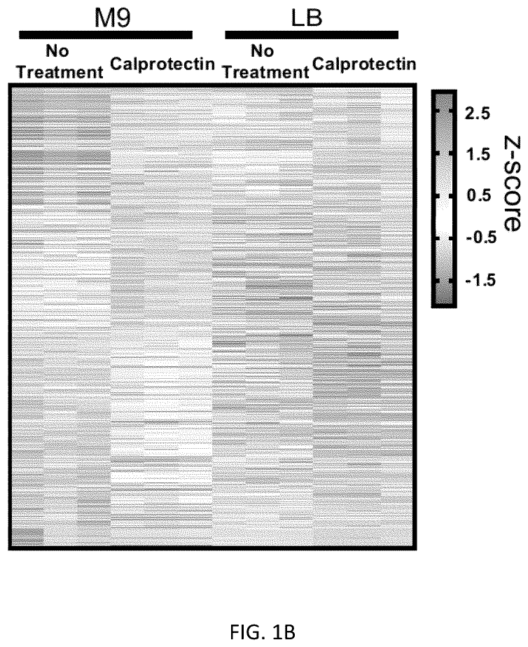

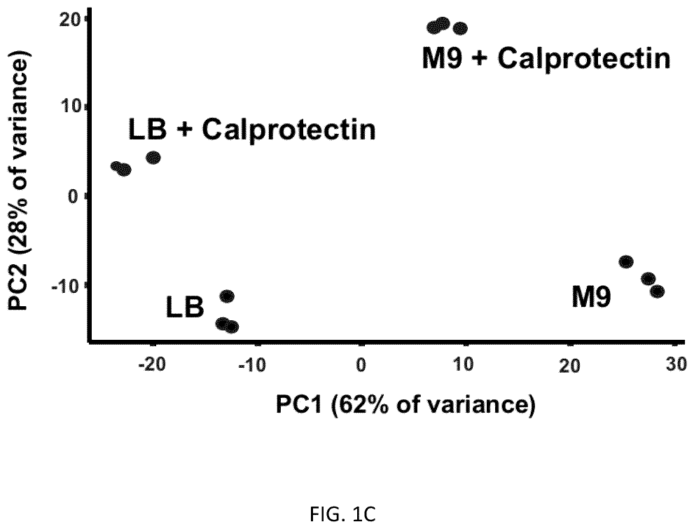

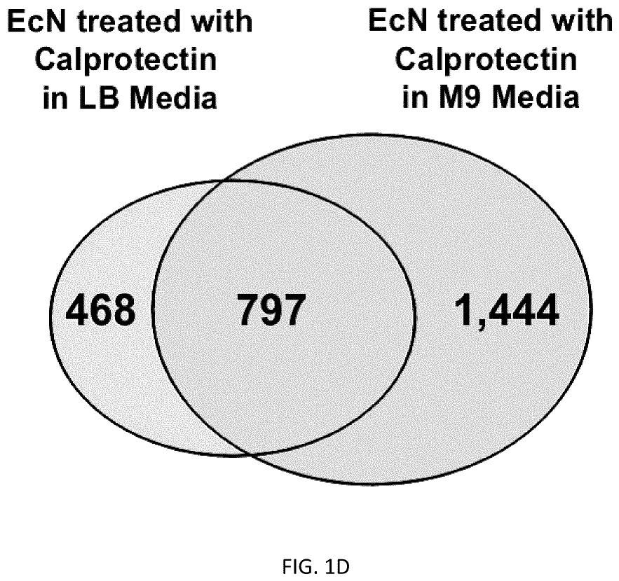

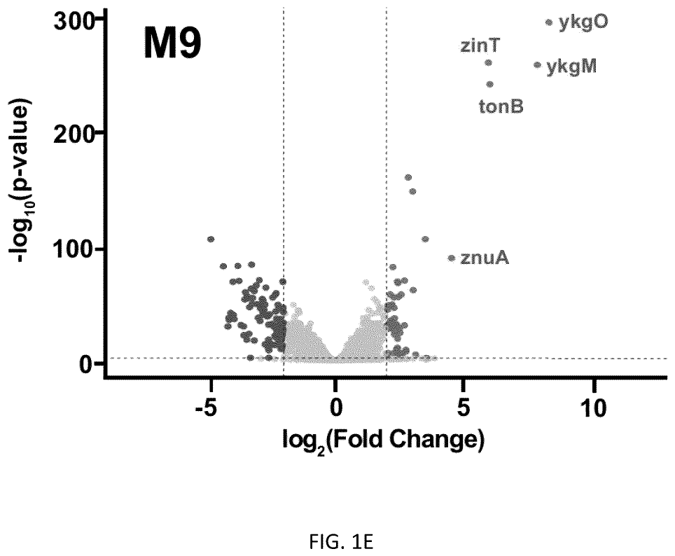

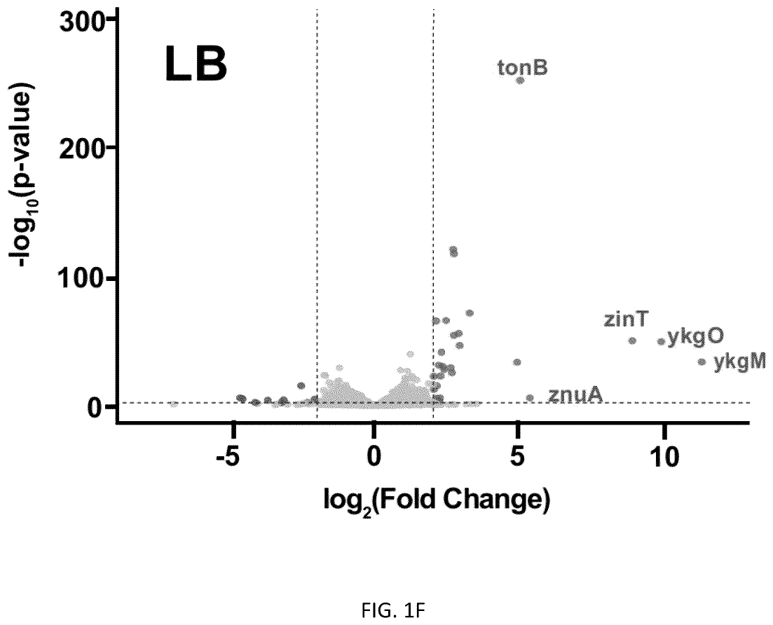

Calprotectin, also known as S100A8/A9, is a heterodimer protein produced by neutrophils and monocytes that exerts antimicrobial activity through chelation of essential nutrients such as zinc and manganese, resulting in bacterial metal starvation. Whether engineered bacterial sensors can be used to detect calprotectin is currently unknown as it is unclear if calprotectin elicits any transcriptional changes in bacterial species. To test whether the human probiotic strain EcN is transcriptionally responsive to calprotectin, EcN was treated with a clinically significant dose of 100 μg/g of human calprotectin and RNA-sequencing (RNA-seq) in the environments of complex (LB) and minimal media (M9) was performed (FIG. 1A). Heatmaps of the statistically significant (padj<0.05) differentially expressed genes that overlapped between calprotectin treated EcN in M9 and LB media were generated (FIG. 1B). A more robust transcriptional response pattern in calprotectin treated EcN in minimal media (M9) as compared to complex media (LB) was observed. Principle component analysis reveals that the global gene expression profile of EcN treated with calprotectin is distinct from untreated in both complex (LB) and minimal (M9) media conditions (FIG. 1C). Overall, there were 2,241 differentially regulated transcripts from calprotectin treated EcN in M9 media compared to 1,265 in LB media, with 797 overlapping genes (FIG. 1D). These results show that calprotectin elicits a robust transcriptional response in EcN that could be used for the construction of the synthetic sensor gene circuit in the probiotic based whole-cell biosensor.

Functional classification of differentially regulated genes by gene ontology (GO) analysis indicated enrichment of genes in pathways involved in ion transport, cellular metabolic processes, and cell motility. Interestingly, the upregulated genes with the highest fold-change were involved in cellular regulation of zinc ion (Zn). This includes the expression of genes ykgM and ykgO, both under the control of the same promoter ykgMO, encoding the zinc-responsive 50S ribosomal subunit proteins L31 and L36, which are increased by approximately 2,500-fold and 1,000-fold respectively in the presence of calprotectin. Fold changes in gene expression after calprotectin treatment appeared to be more robust in EcN grown in minimal media (M9) compared to complex media (LB). Five genes with the both the highest fold-change and statistical significance after calprotectin treatment were identified that were consistent between the 2 groups (FIG. 1E, F). The promoters of these genes were then screened as potential targets to be used for construction of the biosensor of calprotectin.

The Zinc Responsive EcN Promoter ykgMO can Reliably Sense Elevations in Calprotectin In Vitro

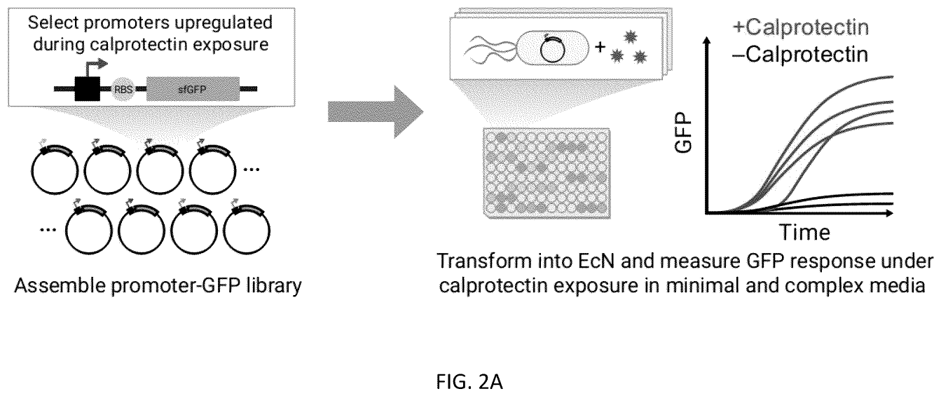

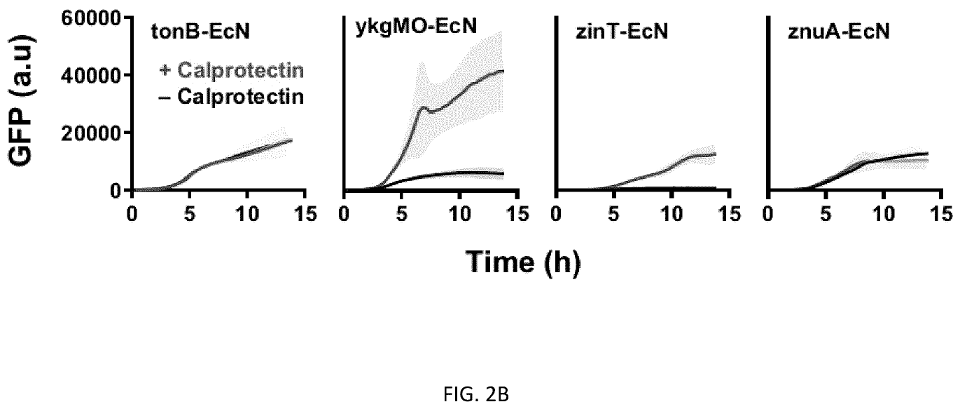

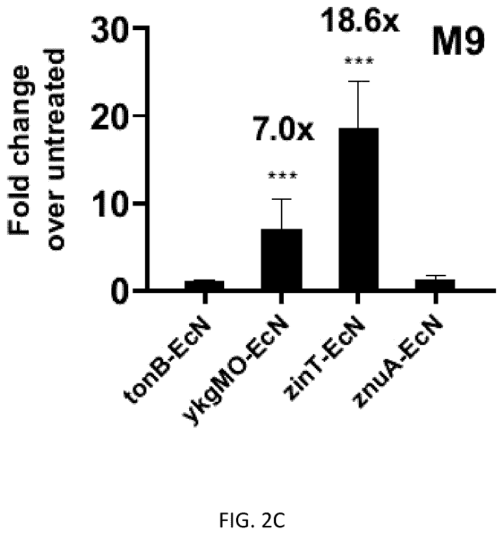

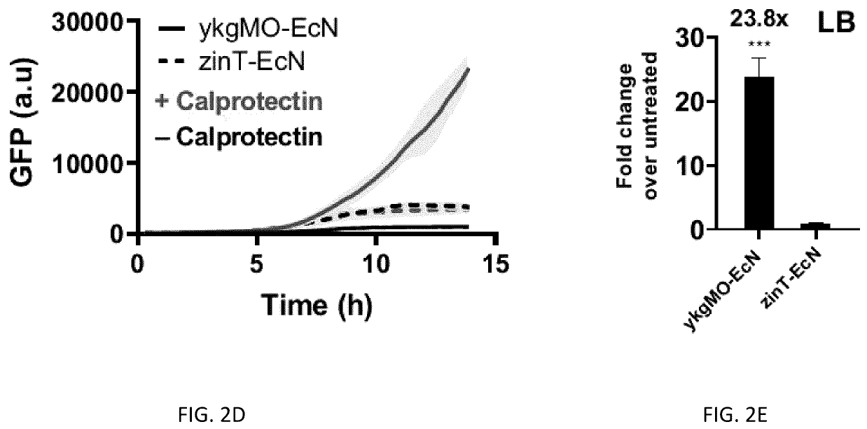

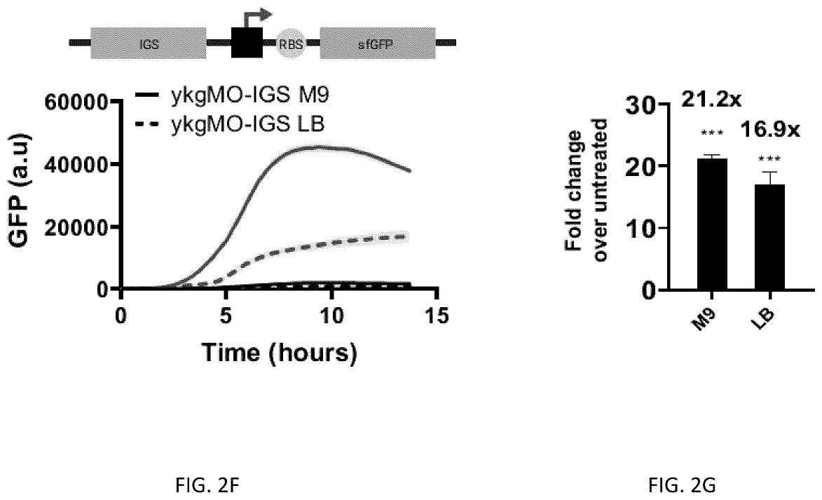

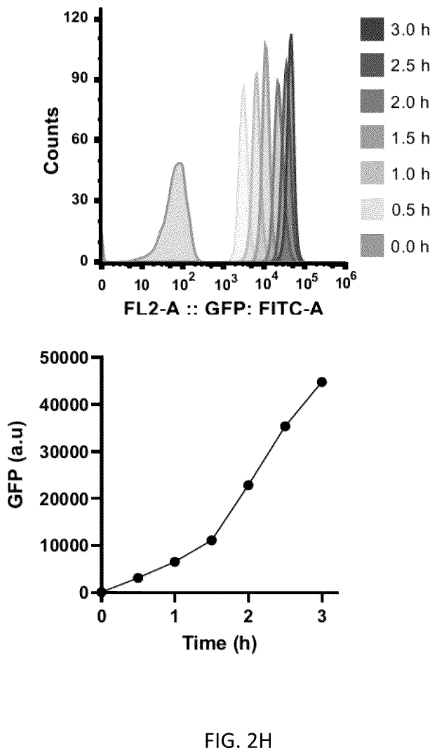

To identify potential biosensors for calprotectin, the respective promoters of the five most significantly upregulated genes were coupled to super folder green-fluorescent reporter protein (sfGFP) prior to transformation into EcN. These recombinant strains of EcN were then treated with 100 μg/g calprotectin to assess for sensitivity of these promoters to calprotectin using sfGFP fluorescence intensity as a surrogate marker (FIG. 2A). Promoters of the top upregulated calprotectin-responsive genes ykgM, ykgO, zinT, znuA, and tonB, which are all induced in the setting of zinc starvation, were initially screened. Interestingly, the expression of these genes is regulated by transcription factors from the ferric uptake regulator family that are responsible for cellular homeostasis of metal ions including manganese, nickel, zinc, and iron. The promoters of ykgMO, zinT, and znuA are all under the control of the transcriptional repressor zinc uptake regulator (Zur) while the promoter of tonB is regulated by the transcription factor ferric uptake regulator (Fur). These results are consistent with a mode of action for calprotectin sensing involving zinc chelation leading to metal starvation.

The responses of biosensors constructed from these promoters in minimal media (M9) were investigated with the goal of identifying promoters with high sensitivity to calprotectin and low background induction during growth phase. EcN transformed with plasmid containing promoters ykgMO (ykgMO-EcN) and zinT (zinT-EcN) demonstrated calprotectin-dependent activation of sfGFP fluorescence compared to untreated controls with similar growth patterns (FIG. 2). ZinT-EcN displayed an 18-fold fluorescence change above untreated controls after 13.8 hours of calprotectin induction, while ykgMO-EcN exhibited a 7-fold change compared to untreated controls (FIG. 2C). The total sfGFP fluorescence of calprotectin-treated ykgMO-EcN at the end of the 13.8 hours growth curve was approximately 4-fold of zinT-EcN (FIG. 2B). However, untreated ykgMO-EcN exhibited 10-fold increase sfGFP fluorescence compared to its zinT-EcN counterpart, suggesting the presence of gene induction during normal cellular growth (FIG. 2B). EcN transformed with plasmid containing promoters for znuA and tonB did not show significant increases in fluorescence compared to untreated controls upon calprotectin treatment. (FIG. 2B). In addition, total sfGFP fluorescence of znuA-EcN and tonB-EcN during the growth curve study was elevated to a comparable degree as zinT-EcN, suggesting significant induction of these promoters during normal cellular growth (FIG. 2B). Thus, only ykgMO-EcN and zinT-EcN remained as viable candidates for the sensor.

Next, it was evaluated whether calprotectin sensitivity in ykgMO-EcN and zinT-EcN was also observed in complex media conditions (LB). When treated with calprotectin in LB, zinT-EcN no longer exhibited its robust calprotectin-dependent activation of sfGFP (FIG. 2D), while ykgMO-EcN maintained its response with 23-fold increase sfGFP fluorescence compared to untreated control (FIG. 2E). Growth conditions were similar for all groups in this study. Interestingly, untreated ykgMO-EcN did not exhibit a similar increase in sfGFP-induction compared to when grown in minimal media after 13.8 hours. This effect is likely due to the lack of metal ion starvation induced gene expression of ykgMO in complex media compared to minimal media conditions.

The intergenic region upstream of genes can contain a variety of functional and regulatory elements, including binding sites for transcription factors. In addition, the zinc-specific regulator, Zur, binds to sites in the intergenic region upstream of known promoter sites of other zinc-responsive ribosomal proteins, like L31/L36 encoded by the ykgMO promoter. Thus, to improve the stability and reduce background expression levels of the ykgMO promoter, the entire intergenic region upstream of the ykgMO gene was identified and coupled with sfGFP to produce a new strain of calprotectin sensing EcN (ykgMO-IGS EcN). This new strain exhibited a 68% reduction in background sfGFP expression level when untreated in minimal media, while maintaining similar induction when treated with calprotectin (FIG. 2F). Sensitivity to calprotectin in complex media for ykgMO-IGS-EcN was comparable to calprotectin-treated ykgMO-ECN (FIG. 2G). Growth conditions were similar for all groups in these experiments. Changes in fluorescence can be detected as early as 1-hour post-treatment of calprotectin in ykgMO-IGS EcN compared to untreated (FIG. 2H). A dose response curve using ykgMO-IGS-EcN strain with varying doses of calprotectin was performed. The lower limit of detection was 25 μg/g of calprotectin and maximum sfGFP signal increased with higher doses of calprotectin. Taken together, these results show that ykgMO-IGS-EcN is a viable candidate for in vivo sensing of calprotectin.

### Calprotectin-Sensing EcN can Reliably Detect Gut Mucosal Inflammation In Vivo

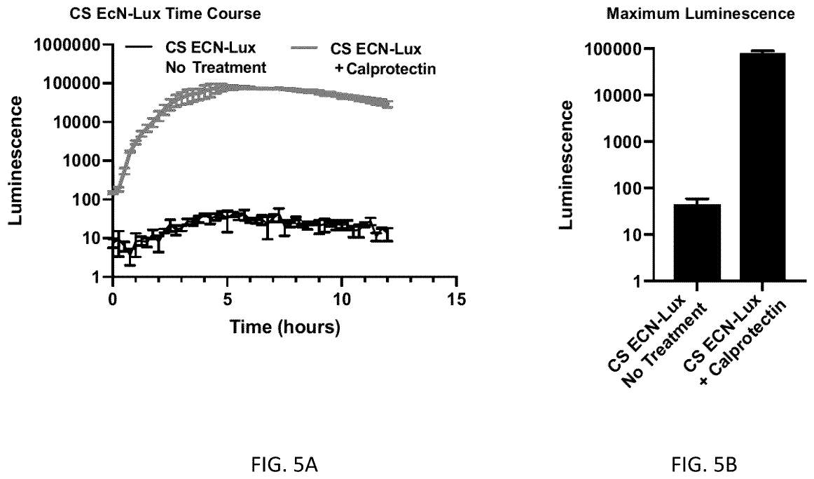

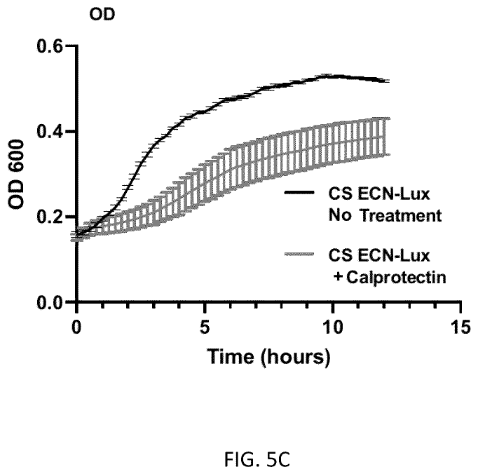

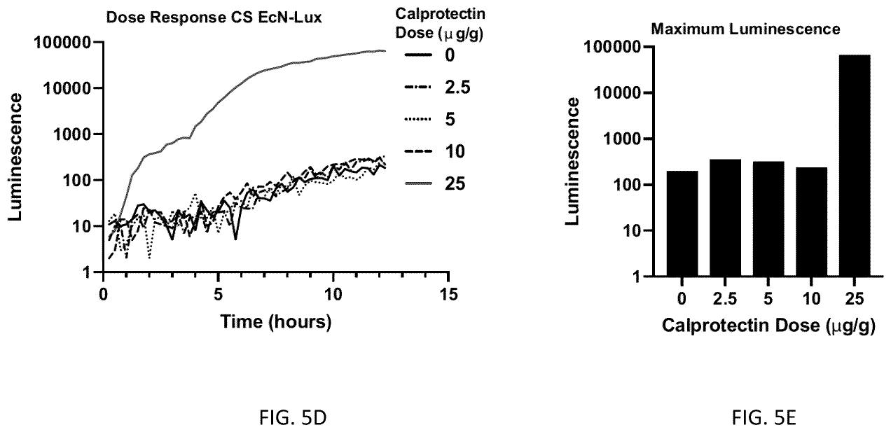

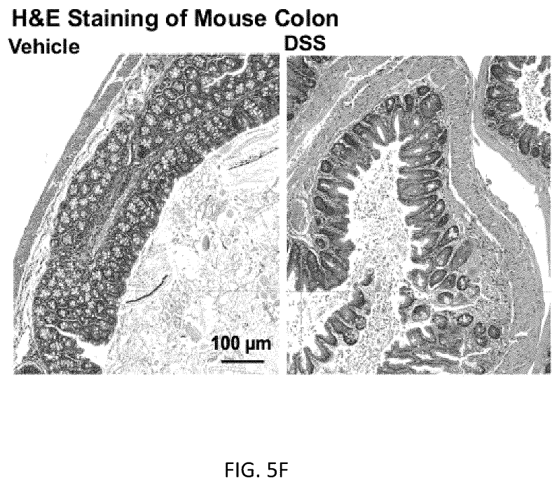

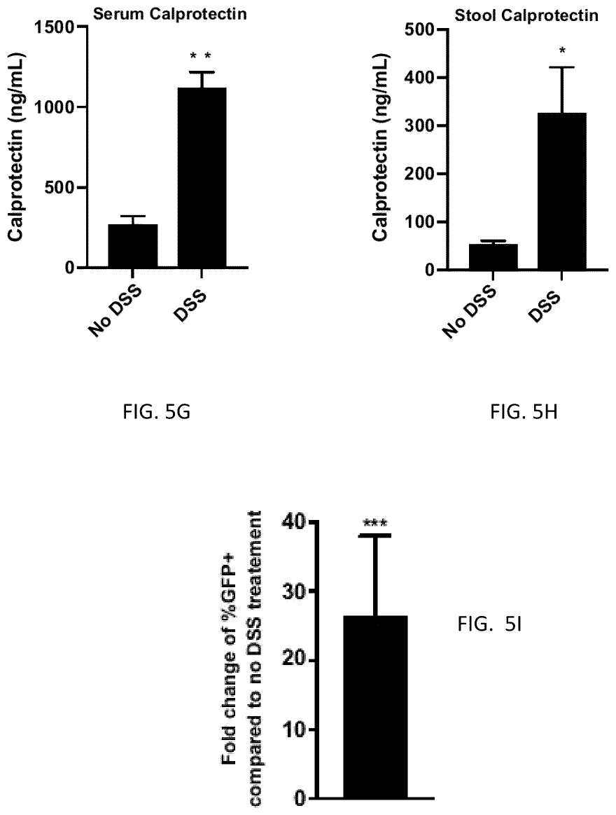

To test whether the calprotectin sensor detects gut inflammation in vivo, the ykgMO-IGS promoter was coupled with luxCDABE cassette and transformed to EcN to generate a new inducible bioluminescent calprotectin-sensing EcN (CS EcN-Lux). This strain of EcN is capable of endogenous production of bacterial luciferin and luciferase to generate a luminescent signal on whole-animal imaging when the ykgMO-IGS promoter is induced. CS EcN-Lux demonstrated robust calprotectin-dependent activation of luminescence compared to untreated controls with similar growth patterns (FIGS. 5A&B). The maximum luminescence of calprotectin treated CS EcN-Lux was 1,830 fold over untreated controls (FIG. 5C). The lower limit of detection of CS EcN-Lux is same as previous experiments from ykgMO-IGS-EcN strain at 25 μg/g of calprotectin (FIGS. 5 D&E).

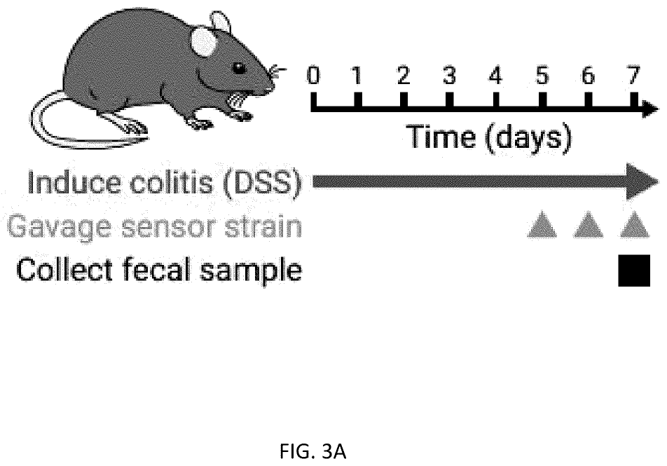

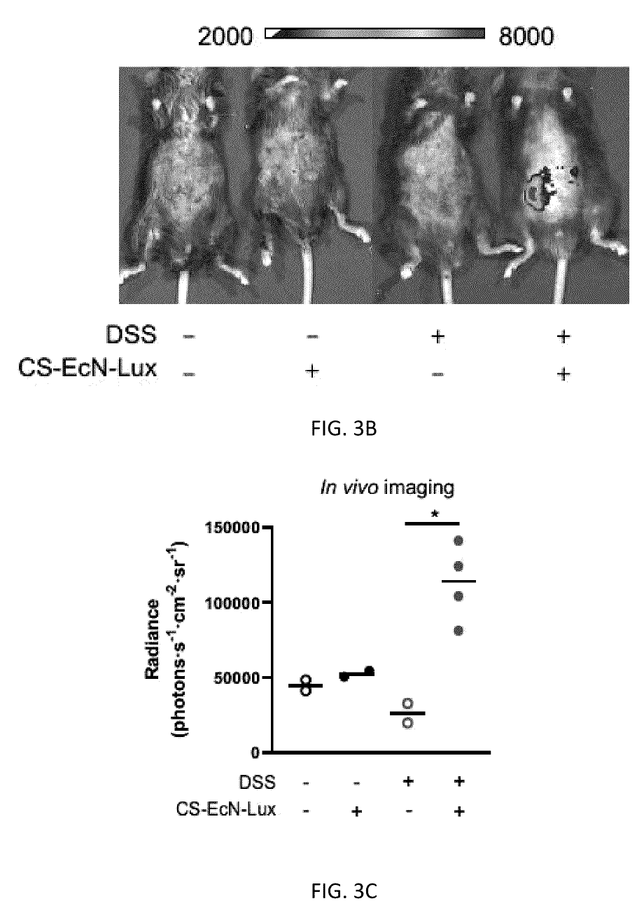

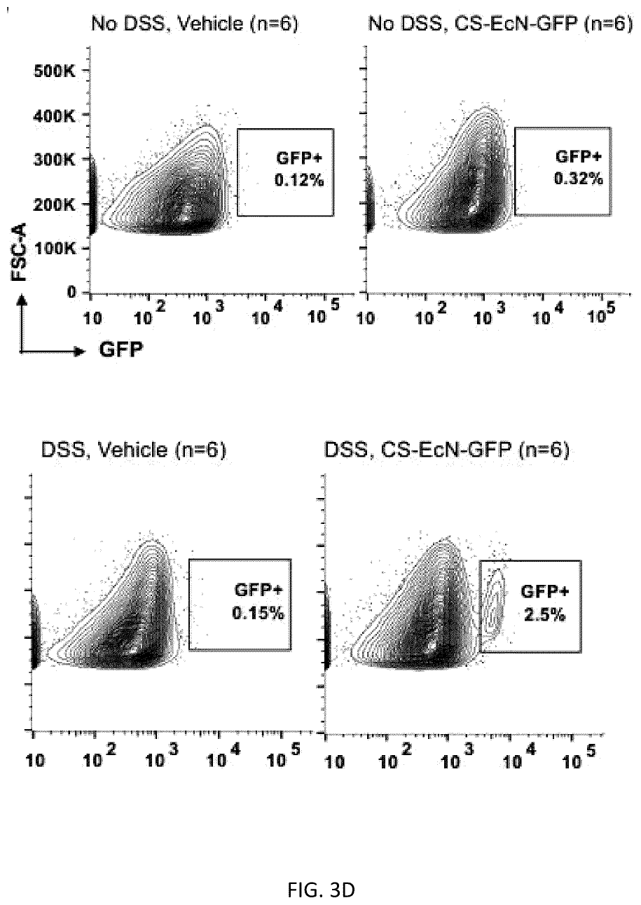

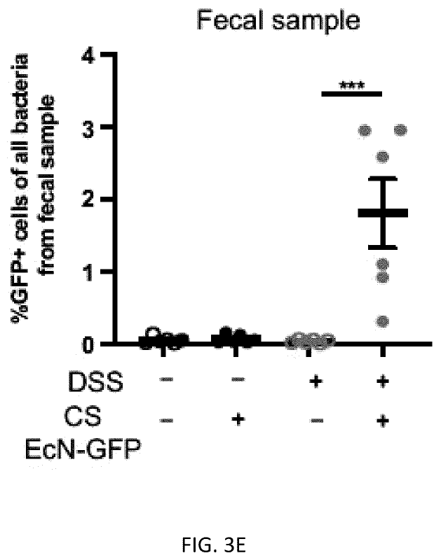

To model inflammatory bowel disease in mice, the dextran sulfate solution (DSS) induced colitis model where mice were given an oral gavage of 3% DSS daily for 7 days (FIG. 3A) was used. The DSS induced colitis model was chosen due its ability to rapidly induce colitis phenotype and given that DSS treatment results in elevation of fecal calprotectin levels. Starting on day 5, control and DSS-treated groups were orally gavaged with either 1×10⁹ CS EcN-Lux or EcN without sensor daily (FIG. 3A). In vivo luminescence imaging was performed using in vivo imaging system (IVIS) on day 7. Luminescent signals were detected in DSS treated mice that were gavaged with CS EcN-Lux with signals reaching 1.13×10⁵ radiance (photons s⁻¹ cm⁻² sr⁻¹) that was significantly increased compared to those measured from mice not treated with DSS and not gavaged CS EcN-Lux (FIG. 3B & FIG. 5F). Mouse colon collected after 7 days of DSS treatment had altered colonic architecture, goblet cell loss, and immune cell infiltration compared to controls, confirming the presence of gut inflammation (FIG. 5G). Serum and stool calprotectin levels were significantly elevated in DSS treated mice as well (FIGS. 5H&I) further confirming that luminescent signals are corresponding elevated calprotectin and gut inflammation caused by DSS treatment.

To further demonstrate that the calprotectin sensor is capable of functioning in the complex environment of the mammalian gut, the ykgMO-IGS-sfGFP reporter strain transformed into EcN, termed CS EcN-GFP, was used for further in vivo studies. Mice were treated with 3% DSS daily and was gavaged with either 1×10⁹ CS EcN-GFP or EcN without sensor at day 5, 6, and 7 of DSS treatment. Stool samples and mouse colons were collected on day 7 (FIG. 3A). Although EcN is a known colonizer of the human GI tract, it is a poor colonizer of the mouse gut. However, EcN can persist transiently in mice pre-treated with antibiotics to reduce local competition. Therefore, carbenicillin was given 2 days prior to bacterial gavage to allow CS EcN-GFP to reach high density. Flow cytometry analysis of stool samples of mice treated with DSS and gavaged with CS EcN-GFP revealed the presence of a distinct sfGFP-positive population of bacteria (FIG. 3D). This sfGFP-positive population was not seen in the absence of DSS or in mice that were not given CS EcN-GFP (FIGS. 3E & F). Furthermore, DSS treated mice that were fed CS EcN-GFP had ˜26-fold increase in this sfGFP-positive bacterial population in stool seen on flow cytometry compared to non-DSS treated mice that were also fed CS EcN-GFP (FIG. 5J). This demonstrates that CS EcN-GFP is highly specific for calprotectin even in a highly heterogeneous environment such as the mouse colon. Thus, both sensor strains are activated in response to gut inflammation in the mouse colon and this signal can be detected in stool samples.

Calprotectin-Sensing EcN can Differentiate Stool Samples from Patients with Active IBD and Those in Remission

To evaluate whether CS EcN-GFP can identify patients with active IBD, fecal samples were collected from patients undergoing stool studies either at the emergency department, in the hospital, and in outpatient clinic settings (FIG. 4A). Stool specimens were collected from 17 patients that consisted of 11 patients with a history of IBD and 6 with no history of IBD (Table 1). Of those 11 patients with IBD, four had a history of Crohn's disease and seven had a history of ulcerative colitis; fecal calprotectin levels were ordered for these patients either for the indication of disease surveillance, therapeutic drug monitoring or suspicion of acute IBD flare. For IBD medications, patients with in the active IBD and IBD in remission groups were on a mix of therapy ranging from oral 5-Aminosalicylic acid to intravenous biologics. Of note, many of the patients also were taking oral multi-vitamin at the time of stool sample collection which includes 5-10 mg of zinc supplementation. Clinical Laboratory quantified calprotectin levels obtained retrospectively from electronic medical records showed that six patients had active IBD with calprotectin ranging from 325-2610 μg/g (Table 1) while those with inactive disease had calprotectin levels ranging from 11-81 μg/g. Of the patients with no IBD history, fecal calprotectin levels were evaluated for indications of diarrhea, rectal bleeding, or abdominal pain with laboratory results of calprotectin levels being <50 μg/g for all specimens. This cohort was used with CS EcN-GFP to discriminate stool samples from patients with active IBD, IBD in remission, and healthy controls.

TABLE 1
Demographics of patients with active IBD, IBD in
remission and no IBD where stool samples were collected

| | | | Laboratory | | | Multi-vitamin |
|---|---|---|---|---|---|---|
| | | | Measured | | IBD | on medication |
| | Sex | Age | Calprotectin | IBD Type | Medication | list |
| No Disease-1 | M | 54 | <5 | NA | NA | yes |
| No Disease-2 | M | 41 | 41 | NA | INA | yes |
| No Disease-3 | F | 27 | 13 | NA | INA | no |
| No Disease-4 | F | 30 | <5 | NA | NA | yes |
| No Disease-5 | F | 42 | <5 | NA | NA | yes |
| No Disease-6 | F | 37 | <5 | NA | NA | no |
| IBD in | M | 67 | 43 | Ulcerative Pancolitis | Mesalamine | no |
| Remission-1 | | | | | | |
| IBD in | F | 53 | 81 | Ileocolonic Crohn's | Certolizumab | no |
| Remission-2 | | | | Disease | | |
| IBD in | F | 46 | 63 | UC with left sided | Vedolizumab | yes |
| Remission-3 | | | | colitis | | |
| IBD in | M | 23 | 54 | Ileal Crohn's Disease | Ustekinumab | Ino |
| Remission-4 | | | | | | |
| IBD in | M | 48 | 11 | Unspecified UC | 6-MP, | no |
| Remission-5 | | | | | Mesalamine | |
| Active IBD-1 | M | 33 | 1200 | Ulcerative Proctitis | Mesalamine | no |
| Active IBD-2 | F | 58 | 634 | UC with left sided | Vedolizumab | Ino |
| | | | | colitis | | |
| Active IBD-3 | F | 52 | 325 | Ulcerative | Mesalamine, | \|no |
| | | | | Proctosigmoiditis | Infliximab | |
| Active IBD-4 | F | 44 | 858 | Ileocolonic CD | Ustekinumab | yes |
| Active IBD-5 | F | 48 | 2390 | Crohn's Colitis | Mesalamine | no |
| Active IBD-6 | F | 33 | 2610 | Ulcerative Pancolitis | Infliximab | yes |

To determine whether CS EcN-GFP is activated in response to stool with elevated levels of calprotectin, CS EcN-GFP was co-cultured with stool samples from patients with no IBD, IBD in remission, and active IBD (FIG. 4A). Immediately after co-culture, sfGFP fluorescence normalized to optical density at 600 mm (OD600) was comparable for the samples from patients with no IBD, IBD in remission and active IBD (FIG. 4B). After 12 hours of co-culturing, CS EcN-GFP co-cultured with fecal samples from patients with active IBD had significantly increased sfGFP/OD600 ratios compared to those that were cultured with samples from patients without IBD or IBD in remission (FIG. 4B). Of the patients with active disease, those with the highest levels of calprotectin also had higher sfGFP/OD600 ratios when co-cultured with EcN-lux exhibiting an overall positive correlation with fold changes ranging from approximately 5 to 15 when compared to healthy controls (FIG. 4C). There was no significant difference between sfGFP/OD600 ratios when comparing patients with no IBD history and those with disease in remission (FIG. 4B). Flow cytometry of CS EcN-GFP co-cultured with fecal samples from patients with active disease confirmed the presence of a distinct sfGFP-positive population of bacteria as early as one hour of co-culturing and saturation of sfGFP-positive populations can be seen with stool sample from patients with higher levels of laboratory measured calprotectin by 3 hours of co-culturing (FIG. 4D-E). In addition, there was a faster rate of activation of sfGFP-positive populations for stool samples from patients with active IBD that had higher laboratory measured calprotectin levels (FIG. 4E, Table 1). Overall, this validates CS EcN-GFP can effectively sense elevated calprotectin in human stool samples and it can be used as a reliable and efficient method of disease activity surveillance in IBD patients.

### Discussion

Provided herein is an engineered probiotic that can reliably detect calprotectin, a biomarker of non-invasive gut inflammation, with sensitivity and specificity for the purpose of disease activity monitoring in IBD. Oral delivery of CS EcN-GFP resulted in the presence of a distinct sfGFP+ bacterial population in stool of mice with DSS induced colitis. These sfGFP+ bacteria were not present in mice that were either not treated with DSS or not given an oral gavage of CS EcN-GFP, suggesting that the activation of our sensor is specific to gut inflammation with low basal activity. Furthermore, oral delivery of CS EcN-Lux resulted in significantly increased luminescence signal in mice treated with DSS that can be seen in live imaging with IVIS. Consistent with the GFP-reporter model, there was minimal leakage of luminescence signal seen in mice not treated with DSS or not gavaged with sensor. Data from clinical stool samples showed that sensor activation was only present when co-cultured with stool from patients with active IBD. Importantly, sfGFP intensity of the sensor can quantitively track with laboratory measured levels of fecal calprotectin. Minimal non-specific sensor activation was observed in this heterogenous population of IBD patients where diet and medications were not controlled. In addition, many of the patients both with and without IBD were taking daily multi-vitamin tablets which usually contains 5-10 mg of zinc supplementation. This did not appear to affect the read-out of the sensor as zinc is absorbed in the small bowel. Overall, the data presented herein indicate that the engineered calprotectin sensing probiotic provides for long-term, non-invasive monitoring of disease activity in IBD that would improve early detection of IBD relapses and clinical outcomes by reducing hospitalizations. Major advantages of this platform compared to conventional enzyme-based ELISA assays of calprotectin include simpler data analysis and on-demand assessment of samples as it avoids the extraction step that is required for all ELISA based assays. In addition, specialized lab equipment is not required to use the sensors or run the reactions as it is culture and plate reader based.

The incidence and prevalence of IBD have been increasing at a global level particularly in developing and newly industrialized nations with historically low rates. In addition, accurate detection of relapsing disease is often delayed in these nations due to lack of reliable diagnostic facilities, particularly endoscopy and pathology. The engineered calprotectin probiotic provided herein could play a key role in in these patient populations as it offers a non-invasive, easy to use method of semi-quantitative determination of IBD disease activity that requires a simple fluorescent or luminescent read out. Offering accurate disease monitoring will result in appropriate risk stratification of patients and optimization of medical management and will bridge the gap in health care disparity in these at-risk patient populations.

### Methods

### RNA Isolation

EcN single colonies were grown overnight in either M9 or LB media. Cells were then diluted 1:100 into 5 mL of M9 or LB media and grown to log phase. Cell cultures were then either incubated with 100 μg/mL of calprotectin or remained untreated for 30 minutes. RNA was then isolated using the QIAGEN RNeasy kit (QIAGEN) according to manufacturer's instructions.

### RNA-Sequencing

RNA quality was checked using Bioanalyzer (Agilent) prior to RNA-seq library preparation. RNAs with an RNA integrity number >8 were used for library preparation, which was constructed from 100 ng of RNA with the Illumina Stranded Total RNA Prep, Ligation with Ribo-Zero Plus kit (Illumina). RNA Sequencing was then performed on NovaSeq 6000 sequencer and analyzed as previously described. Reads were aligned to the GCF_003546975.1 assembly of the Escherichia coli Nissle 1917 reference genome with STAR (v2.7.5) using the GeneCounts option. Gene counts were obtained from EcN annotations (Refseq) using rsem-calculate-expression (v.1.3.0). Analysis of differential expression between treatments and media conditions using DESeq2 (v1.32.0) after removal of genes with <10 assigned counts. FDR-adjusted p-value cutoff for significance was set to 0.05. Plots were made in R (v4.0.3) using ggplot2 (v3.3.0), pheatmap (v1.0.12). Annotations were converted to gene IDs using EcoCyc (www.ecocyc.org). Pathway and biological function assignments of differentially expressed genes were completed using DAVID bioinformatics tools (https://david.nciferf.gov) on all statistically significant differentially expressed genes (p<0.05) in EcN treated with calprotectin and untreated controls in both LB and M9 media.

### DNA Cloning

Custom promoter sequences were ordered from Integrated DNA Technologies (IDT) and cloned upstream of sfGFP reporter or luxCDABE cassette in a P15A backbone with chloramphenicol resistance. All plasmid assembly was performed using Gibson Assembly using the Gibson Assembly Master Mix (NEB). The assembled plasmid was transformed into chemically competent EcN and plated onto LB agar with appropriate antibiotics.

### Calprotectin Induction

EcN containing sensor plasmids were grown overnight, diluted 1:100 in either M9 or LB and then treated with 100 μg/mL of calprotectin. SfGFP fluorescence and OD600 were recorded for 12 hours in TECAN Infinite plate reader. Luminescence imaging was quantified using a BioTek Synergy Neo2 Multi-mode plate reader.

### Dextran Sodium Sulfate Mouse Experiments

Colitis was induced in six- to eight-week-old male C57BL/6 mice by administration of 3% (W/V) DSS (MW ˜40,000, Sigma) in drinking water for 7 days. On day 3 of DSS, mice were given an oral gavage of carbenicillin to promote engraftment of probiotic in the mouse gut. On days 5, 6, and 7 of DSS, mice were given an oral gavage of 1×10⁹ CFUs of CS EcN-Lux/CS EcN-GFP or EcN without sensor. Six hours after probiotic sensor gavage on day 7 of DSS, mice were euthanized and stool from proximal and distal colon was collected for sfGFP analysis.

### Histology

Colon tissue of DSS treated and untreated mice were fixed in 4% paraformaldehyde for 24 hours before transfer to 70% ethanol. Paraffin-embedding, sectioning, and hematoxylin and eosin (H&E) staining were performed by Northwestern University Research Histology and Phenotyping Laboratory Core.

### Mouse Fecal Sample Preparation

Mouse stool collected from colon were homogenized in 1.5 mL of sterile PBS. Samples were then vortexed and filtered through Sum syringe filter (Millipore) to remove solid components and host cells. Filtered samples were incubated for 1 hour in 37 Celsius to allow for maturation of fluorophores and then analyzed by flow cytometry.

### Human Stool Sample Collection and Preparation

0.5-1 g of stool sample were collected from stool samples of patients in the emergency department, outpatient clinic, and inpatient unit. Samples were collected only when excess stool samples were available after all necessary clinical laboratory testing were completed. No extra samples were requested from patients. All biospecimens were labeled with a unique subject code that did not contain any personal identifiers.

For co-culturing experiments, we homogenized ˜0.1 g of human stool sample with sterile PBS to make a final concentration 1 mg/μL. If the stool was liquid, homogenization was not done and 100 μL of liquid stool sample was used instead. This mixture was then co-cultured with 2 mL of 1:100 diluted CS EcN in log-phase in M9 media for 12 hours. A 200 μL aliquot of this mixture was then quantified in the TECAN infinite plate reader for OD600 and sfGFP with excitation/emission wavelength set to 485/530 nm and gain set to 100.

### Calprotectin Quantification

Measured calprotectin levels for the patient samples were performed by the Northwestern Clinical Pathology Laboratory. Calprotectin was a send out lab to Quest Diagnostics and quantification was performed at Quest Nichols Institute in Chantilly, VA. Quest Diagnostics utilize an FDA approved chemiluminescence method for calprotectin detection requiring at least 0.3 g of stool sample that has been in storage in room temperature for less than 6 days. No preservatives were used in storing of samples after collection. Calprotectin levels were obtained retrospectively through electronic medical records.

Mouse Calprotectin levels were quantified using ELISA (Immundiagnostik) from serum and stool collected from mice after 7 days of treatment with 3% DSS.

### Flow Cytometry

Flow cytometry was performed using a Sony SH800 cytometer using a standard 408 nm laser configuration and a 100 μm sorting chip. Cells were gated on FSC-A and SSC-A to exclude debris followed by FSC-H and FSC-W to isolate single cells. For pure EcN culture experiments, cells were treated with calprotectin for one hour prior to analysis. For human stool experiments, 1 mL of CS EcN-GFP grown to log-phase was co-cultured with human stool samples for 1-3 hours prior to analysis. Flow cytometry plots were generated with FlowJo (Becton, Dickinson & Company).

### Fluorescence and Luminescence Imaging

SfGFP fluorescence in all our studies were measured using a TECAN Infinite MPLEX plate reader with excitation/emission wavelength set to 485/530 nm and gain set to 100. Luminescence imaging was quantified using a BioTek Synergy Neo2 Multi-mode plate reader with gain set to 135.

### Mouse Experiments

All mice were maintained on a C57BL/6J background. Wildtype C57BL/6J mice were obtained from Jackson Laboratories. Mice were maintained at room temperature with a 12-hour light/dark cycle and free access to water and food. All animal experiments were performed according to procedures approved by the Northwestern University Institutional Animal Care and Use Committee.

### IVIS Imaging

To image in vivo bacterial luminescence, mice were shaved and anesthetized with isoflurane and imaged using IVIS Spectrum Instrument (Perkin Elmer). The bacteria LuxCDABE cassette produced a luminescent signal without provision of an exogenous substrate. Quantification of luminescence was done using the Living Image Analysis Software (Perkin Elmer).

### Statistical Analyses

Statistical tests were calculated in either Microsoft Excel (Student's T-test) or GraphPad Prism 9.0 (ANOVA, Student's T-test). Differences between the two groups over time were determined by a two-way repeated measures ANOVA. For comparisons between two independent groups, a Student's T-test was used. Significance was accepted at p<0.05. The details of the statistical tests carried out are indicated in respective figure legends.

### Example 2

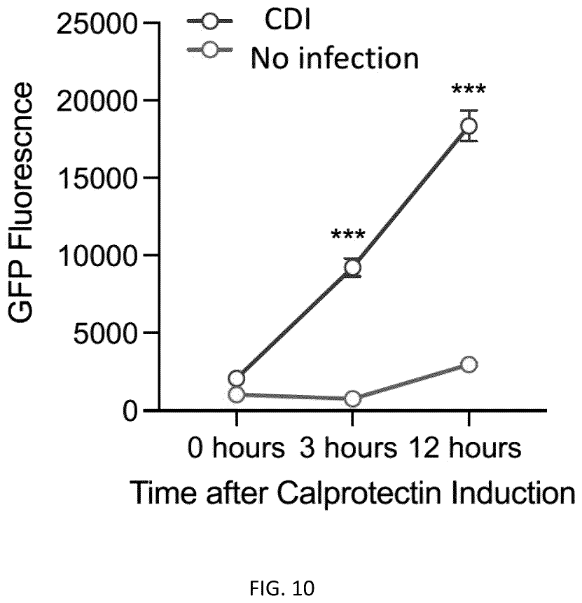

Additional diseases beyond inflammatory bowel disease characterized by mucosal damage and resulting calprotectin production can be detected using the engineered bacterial biosensors described herein. For example, gastrointestinal infections and celiac disease cause mucosal damage and calprotectin production and can also be detected using the biosensors provided herein. Indeed, recombinant EcN was also shown herein to effectively differentiate stool samples from patients with active C. difficile infection (CDI) from those without infection. Fecal samples of patients with CDI and with no gut infection were treated with recombinant EcN and samples were quantified using a TECAN infinite plate reader after 3 hours and after 12 hours. Results are shown in FIG. 10. As shown, recombinant EcN was able to effectively differentiate patients with CDI and patients with no infection at each time point. Celiac disease is another example of a gastrointestinal disease that causes mucosal damage and calprotectin production, which can be detected using a bacterial biosensor provided herein. For example, stool sample obtained from a patient suspected of having Celiac disease can be contacted with a biosensor provided herein and the detectable signal can be quantified to determine whether the subject has Celiac disease.

1. An engineered bacterial biosensor of gastrointestinal disease, the engineered bacterial biosensor comprising a bacterial host cell expressing a reporter molecule operably linked to a calprotectin-responsive promoter.

2. The engineered bacterial biosensor of claim 1, wherein the reporter molecule produces a detectable signal when levels of calprotectin in an environment containing the engineered bacterial biosensor are equal to or above a threshold value.

3. The engineered bacterial biosensor of claim 1, wherein the bacterial host cell comprises a probiotic bacterial strain.

4. The engineered bacterial biosensor of claim 3, wherein the bacterial host cell comprises a probiotic E. coli strain.

5. The engineered bacterial biosensor of claim 4, wherein the bacterial host cell comprises E. coli Nissle 1917 (EcN).

6. The engineered bacterial biosensor of claim 1, wherein the reporter molecule comprises a fluorescent reporter molecule, a bioluminescent reporter molecule, a colorimetric reporter molecule, a CT-detectable reporter molecule, an MRI-detectable reporter molecule, or an ultrasound-detectable reporter molecule.

7. The engineered bacterial biosensor of claim 1, wherein the calprotectin-responsive promoter comprises ykgMO.

8. The engineered bacterial biosensor of claim 1, wherein the calprotectin-responsive promoter is encoded by a polynucleotide having at least 80% sequence identity with SEQ ID NO: 1, SEQ ID NO: 2, SEQ ID NO: 4, or SEQ ID NO: 5.

9. The engineered bacterial biosensor of claim 1, wherein the gastrointestinal disease comprises inflammatory bowel disease, a gastrointestinal infection, or Celiac disease.

10. A method comprising:
a) providing the engineered bacterial biosensor of claim 1 to a subject, obtaining a sample from the subject, and measuring a signal from the reporter molecule in the sample; or
b) contacting a sample obtained from a subject with the engineered bacterial biosensor of claim 1 and measuring a signal from the reporter molecule in the sample, wherein the subject has or is suspected of having a gastrointestinal disease.

11. The method of claim 10, wherein the sample is a stool sample.

12. The method of claim 10, wherein a signal equal to or above a threshold value indicates that the subject has the gastrointestinal disease.

13. The method of claim 10, wherein the gastrointestinal disease comprises inflammatory bowel disease, Celiac disease, or a gastrointestinal infection.

14. The method of claim 13, wherein a signal equal to or above a threshold value indicates that the subject has active inflammatory bowel disease and wherein a signal below a threshold value indicates that the subject is in remission or does not have inflammatory bowel disease.

15. The method of claim 13, wherein the gastrointestinal infection comprises a Clostridium difficile infection.

16. A method comprising:
a) providing the engineered bacterial biosensor of claim 1 to a subject having or suspected of having a gastrointestinal disease; and
b) performing an imaging procedure on the subject to measure a detectable signal from the engineered bacterial biosensor within the subject.

17. The method of claim 16, wherein a signal equal to or above a threshold value indicates that the subject has the gastrointestinal disease.

18. The method of claim 16, wherein the gastrointestinal disease comprises inflammatory bowel disease, Celiac disease, or a gastrointestinal infection.

19. The method of claim 18, wherein a signal equal to or above a threshold value indicates that the subject has active inflammatory bowel disease and wherein a signal below a threshold value indicates that the subject is in remission or does not have inflammatory bowel disease.

20. The method of claim 18, wherein the gastrointestinal infection comprises a Clostridium difficile infection.


SEQUENCE LISTING (18 sequences)

SEQ ID NO: 1 - DNA, 81 bp, Synthetic construct
  source 1..81: mol_type = other DNA
ccacaaagagtcacagggattgagtgttgaaatgatccggatgagcatgtatctttatggttatgttataacataacaggt

SEQ ID NO: 2 - DNA, 364 bp, Synthetic construct
  source 1..364: mol_type = other DNA
taacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatg

SEQ ID NO: 3 - DNA, 12 bp, Synthetic construct
  source 1..12: mol_type = other DNA
aaagaggagaaa

SEQ ID NO: 4 - DNA, 93 bp, Synthetic construct
  source 1..93: mol_type = other DNA
ccacaaagagtcacagggattgagtgttgaaatgatccggatgagcatgtatctttatggttatgttataacataacaggtaaagaggagaaa

SEQ ID NO: 5 - DNA, 376 bp, Synthetic construct
  source 1..376: mol_type = other DNA
taacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatgaaagaggagaaa

SEQ ID NO: 6 - DNA, 717 bp, Synthetic construct
  source 1..717: mol_type = other DNA
atgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataa

SEQ ID NO: 7 - DNA, 30 bp, Synthetic construct
  source 1..30: mol_type = other DNA
aaagaggagaaatactagatgcgtaaaggc

SEQ ID NO: 8 - DNA, 53 bp, Synthetic construct
  source 1..53: mol_type = other DNA
tatgttataacataacaggtaacaaagaggagaaatactagatgcgtaaaggc

SEQ ID NO: 9 - DNA, 220 bp, Synthetic construct
  source 1..220: mol_type = other DNA
ggtggcttaatgtttccatcgaagcactatattggtgcaacattcacatcgtcgttgtgcagcccttttgcacgatggtgcccacaaagagtcacagggattgagtgttgaaatgatccggatgagcatgtatctttatggttatgttataacataacaggtaacaaagaggagaaatactagatgcgtaaaggcgaagagctgttcactggtgtcgtcc

SEQ ID NO: 10 - DNA, 218 bp, Synthetic construct
  source 1..218: mol_type = other DNA
ccaccgaatataaaggtagcttcgtgataaaccacgttgtaagtgtagcagcaacacgtcgggaaaacgtgctacaacgggtgtttctcagtgtccctaactcacaactttactaggcctactcgtacatagaaatacaatacaatattgtattgtccattggtttctcctctttatgatctacgcatttccgcttctcgacaagtgaccacagcagg

SEQ ID NO: 11 - DNA, 24 bp, Synthetic construct
  source 1..24: mol_type = other DNA
tgatattgccgttatttgacaagt

SEQ ID NO: 12 - DNA, 72 bp, Synthetic construct
  source 1..72: mol_type = other DNA
tataacataacaggtaaaaatgaaagaggagaaaggatctatgaaatttggcaactttttgcttacatacca

SEQ ID NO: 13 - DNA, 438 bp, Synthetic construct
  source 1..438: mol_type = other DNA
actataacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatccacaaatagtcacagtgattgggtgttgaaatgatccggatgagcatgtatctttacggttatgttataacataacaggtaaaaatgaaagagagaaaggatctatgaaatttggcaactttttgcttacataccaacctccccaattttctcaaaca

SEQ ID NO: 14 - DNA, 438 bp, Synthetic construct
  source 1..438: mol_type = other DNA
tgatattgccgttatttgacaagtgaagtcactataaattttatacgtaggagagggaaaaaacattcattaataatataggcaccctctccttatgtgtaacagtccattagttagtacgacgttatttagtagccggtcatttcacctctatcggaggtaagagctttttaggtatgagagtcgctttggtagtagttagtgagtaggtccgcaaataccctcgcagcggttaccgacgattgttacggtctgaaggggcaacgcctttaaggtgtagggtgtttatcagtgtcactaacccacaactttactaggcctactcgtacatagaaatgccaatacaatattgtattgtccatttttactttctcctctttcctagatactttaaaccgttgaaaaacgaatgtatggttgaggggttaaaagagttgt

SEQ ID NO: 15 - DNA, 414 bp, Synthetic construct
  source 1..414: mol_type = other DNA
ttcacatcgtcgtggtgcagcccttttgcacgatggtgctaacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatcaagaggagaaa

SEQ ID NO: 16 - DNA, 416 bp, Synthetic construct
  source 1..416: mol_type = other DNA
aagtgtagcagcaccacgtcgggaaaacgtgctaccacgattgccgttatttgacaagtgaagtcactataaattttatacgtaggagagggaaaaaacattcattaataatataggcaccctctccttatgtgtaacagtccaattagttagtacgacgttatttagtagccggtcatttcacctctatcggaggtaagagctttttaggtatgagagtcgctttggtagtagttagtgagtaggtccgcaaataccctcgcagcggttaccgacgattgttacggtctgaaggggcaacgcctttaaggtgtagggtgtttatcagtgtcactaacccacaactttactaggcctactcgtacatagaaatgccaatacaatattgtattgtccatttttactttctcctcttt

SEQ ID NO: 17 - DNA, 92 bp, Synthetic construct
  source 1..92: mol_type = other DNA
ccacaaagagtcacagggattgagtgttgaaatgatccggtgagcatgtatctttatggttatgttataacataacaggtaaagaggagaaa

SEQ ID NO: 18 - DNA, 93 bp, Synthetic construct
  source 1..93: mol_type = other DNA
ggtgtttctcagtgtccctaactcacaactttactaggcctactcgtacatagaaataccaatacaatattgtattgtccatttctcctcttt